SEQUENCE LISTING (13 sequences)

SEQ ID NO: 1 - DNA, 1337 bp, Homo sapiens
  CDS (9)...(1073)
ccccgaccatggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaacagaccgatttgcacccatgccatagaaactgaggccattatttcaaccgcttgccatattccgaggacccagtgttcttagctgaacaatgaatgctgaccctaaatgtggacatcatgaagcatcaaagcactgtttaactgggagtgatatgatgtgtagggcttttttttgagaatacactatcaaatcagtcttggaatacttgaaaacctcatttaccataaaaatccttctcactaaaatggataaatcagtta

SEQ ID NO: 2 - protein, 355 aa, Homo sapiens [sequence omitted]

SEQ ID NO: 3 - DNA, 3535 bp, Rattus norvegicus
  CDS (30)...(1538)
cgctttggaaattttcctgcttttgcaaaatgatgactatttctttgatttggggaattgccgtgttggtgagctgttgcatatggtttattgttggaataaggagaaggaaagctggtgaacctcctttggagaacgggttgattccgtacctgggctgtgctctgaaatttggatctaatcctcttgagttcctaagagctaatcaaaggaagcatggtcacgtttttacctgcaaactgatggggaaatatgtccatttcatcacaaactccctgtcataccacaaagtcttatgtcatggaaaatattttgactggaaaaaatttcattacactacttctgcgaaggcatttggacacagaagcattgacccaaatgatggaaataccacggaaaatataaacaacacttttaccaaaaccctccagggagatgctctgtgttcactttctgaagccatgatgcaaaacctccaatctgtcatgagacctcctggccttcctaaatcaaagagcaatgcctgggtcacggaagggatgtatgccttctgttaccgagtgatgtttgaagctggctatctaacactgtttggcagagatatttcaaagacagacacacaaaaagcacttattctaaacaaccttgacaacttcaaacaatttgaccaagtctttccggcactggtggcaggccttcctattcacttgttcaagaccgcacataaagctcgggaaaagctggctgagggattgaagcacaagaacctgtgtgtgagggaccaggtctctgaactgatccgtctacgtatgtttctcaatgacacgctctccacctttgacgacatggagaaggccaagacgcacctcgctattctctgggcatctcaagcaaacaccattcctgcaaccttttggagcttatttcaaatgatcaggagtcctgaagcaatgaaagcagcctctgaagaagtgagtggagctttacagagtgctggccaagagctcagctctggagggagtgccatttacttggatcaagtgcaactgaatgacctgccggtactagacagcatcatcaaggaggctctgaggctttccagtgcatccttgaatatccgcacagctaaggaggacttcactctccatcttgaggacggttcctataacatccgaaaagatgacatgatagctctttatccacagttaatgcacttggatcctgaaatctacccagaccctttgactttcaaatatgaccggtaccttgatgaaagcgggaaagcaaagaccaccttctacagtaatggaaacaagctgaagtgtttctacatgcccttcggatcaggcgcgacaatatgtcctggaagactctttgccgtccaagaaatcaagcagtttttgatcctgatgctctcctgctttgaactggagtttgtggagagccaagtcaagtgtccccctctagaccagtcccgggcaggcttgggaattttgccaccactacatgatattgagtttaaatataaactgaaacactgatacgtggttggaagaagcgaacactggatgatgtcacttggcggctgagagtcatcactaaacaggccttcgggaccaatgctcactgatgcgccctagcgactggattagtgggaagaactttgttctcgctgcccacattcctgggtgttcacatagctggggccagagcttcatcactttcagaaagcaatgtcttttgtatttattttcaaaatgaagatattccaattggcaggatatttttcctaaggaaattgctttatatttttatgaaaactaccaattaattatgaaagggcttgaaattcacgttttagtgaaattactgatttttcactagtaaggttcttcaggtgtgaaactgtattataaaaatgttgtaatgggtcacactgtgctttgcataaaggtaaaggaaactatgtttcagccttttctgtgtctatgagcttcgaaaataatcttactgttctagaaacactggggaggtttcgacatgctctcgctatattttattttactgttgctagaaattttcattccagttttcaactaccttatctttcccccattttgacatgcatgccaatgagaagagtattttttaggaattaacaaggcacctcccagaaccctaccctgagacttttaagcctttaatcccagcactcgagaagtagagccaggcagatctctgagtctgaggttattctggtctacatcagctccagacaagccaggactacagaatgggatcttgtctaaaaaatacagctaatctttatgtcataactgattatgaatcaacctaaaagataaattttcaatcaggactcagagaaaatgagcaattaaaaaacttagctctgaggtatgtggaattcattaagtacaagttgacattacatgttctttaaaaatagtttatgttttatctctaaatgccctgcagatgaagaataataatgaaaagttgaataatactgtttaaacactaagtgcaataatgctttggtaatgtactttaagagaatcattagccgtgccagttttactaaaatatatttatatgtaaattatatttatctttttcttataccataaatataaaaatattgcaacatttagtaattttaaaattatatacctttcagaaaatgatgtatgatgtttgtatgttttttaactttgaacagaacatttaaattattcatctacggtgatttttatcttatttatttctttttgtctcattcatatcttgaagaaatccaaaaatatctgaaggaatcgctcactcaaatgtctccctatggttacagaaaaattcaataccatgtttttgtcctcggggactgaagcagggtgtcgtgggtgcgagcagaggctcctgctgcagcgagctttatccacgggactccttaaacttttaaaatcttatcactattatcatgcatttattacctaagtaggatatttccctttcctttttcatttcagcagagtcccttagcaacccaggctgactgggaccctccatgtagcttaagctgtgaactcactgtacttcctgttttcacttattttaggaagtaattttccctatcagaaattttaattgtttagatgatgtataagagtaacacaattctgttatatactaatctgtagtaaactaaatttgttcttagaacaagtttgatgactctcaaattgaatgtatccatacatctttccatggcttcttgaatgcccatttctcatacacagaatgatgggtttcacggtgatgtcttcctttcatgtctttattcttgtgcggtgatggttggcaaatgatacccatggagcaaggttactcttcctatttctgtgcagcctaagtgttaagaataatttttaaatacttggagggaaggcacattttgtgtcatatgtgaagtgacatgtgacacacagactagcaaatccatgagtaaaattttattggg

SEQ ID NO: 4 - protein, 503 aa, Rattus norvegicus
MMTISLIWGIAVLVSCCIWFIVGIRRRKAGEPPLENGLIPYLGCALKFGSNPLEFLRANQRKHGHVFTCKLMGKYVHFITNSLSYHKVLCHGKYFDWKKFHYTTSAKAFGHRSIDPNDGNTTENINNTFTKTLQGDALCSLSEAMMQNLQSVMRPPGLPKSKSNAWVTEGMYAFCYRVMFEAGYLTLFGRDISKTDTQKALILNNLDNFKQFDQVFPALVAGLPIHLFKTAHKAREKLAEGLKHKNLCVRDQVSELIRLRMFLNDTLSTFDDMEKAKTHLAILWASQANTIPATFWSLFQMIRSPEAMKAASEEVSGALQSAGQELSSGGSAIYLDQVQLNDLPVLDSIIKEALRLSSASLNIRTAKEDFTLHLEDGSYNIRKDDMIALYPQLMHLDPEIYPDPLTFKYDRYLDESGKAKTTFYSNGNKLKCFYMPFGSGATICPGRLFAVQEIKQFLILMLSCFELEFVESQVKCPPLDQSRAGLGILPPLHDIEFKYKLKH

SEQ ID NO: 5 - DNA, 2850 bp, Homo sapiens
  CDS (40)...(1551)
agattttcttcctcagagattttggcctagatttgcaaaatgatgaccacatctttgatttgggggattgctatagcagcatgctgttgtctatggcttattcttggaattaggagaaggcaaacgggtgaaccacctctagagaatggattaattccatacctgggctgtgctctgcaatttggtgccaatcctcttgagttcctcagagcaaatcaaaggaaacatggtcatgtttttacctgcaaactaatgggaaaatatgtccatttcatcacaaatcccttgtcataccataaggtgttgtgccacggaaaatattttgattggaaaaaatttcactttgctacttctgcgaaggcatttgggcacagaagcattgacccgatggatggaaataccactgaaaacataaacgacactttcatcaaaaccctgcagggccatgccttgaattccctcacggaaagcatgatggaaaacctccaacgtatcatgagacctccagtctcctctaactcaaagaccgctgcctgggtgacagaagggatgtattctttctgctaccgagtgatgtttgaagctgggtatttaactatctttggcagagatcttacaaggcgggacacacagaaagcacatattctaaacaatcttgacaacttcaagcaattcgacaaagtctttccagccctggtagcaggcctccccattcacatgttcaggactgcgcacaatgcccgggagaaactggcagagagcttgaggcacgagaacctccaaaagagggaaagcatctcagaactgatcagcctgcgcatgtttctcaatgacactttgtccacctttgatgatctggagaaggccaagacacacctcgtggtcctctgggcatcgcaagcaaacaccattccagcgactttctggagtttatttcaaatgattaggaacccagaagcaatgaaagcagctactgaagaagtgaaaagaacattagagaatgctggtcaaaaagtcagcttggaaggcaatcctatttgtttgagtcaagcagaactgaatgacctgccagtattagatagtataatcaaggaatcgctgaggctttccagtgcctccctcaacatccggacagctaaggaggatttcactttgcaccttgaggacggttcctacaacatccgaaaagatgacatcatagctctttacccacagttaatgcacttagatccagaaatctacccagaccctttgacttttaaatatgataggtatcttgatgaaaacgggaagacaaagactaccttctattgtaatggactcaagttaaagtattactacatgccctttggatcgggagctacaatatgtcctggaagattgttcgctatccacgaaatcaagcaatttttgattctgatgctttcttattttgaattggagcttatagagggccaagctaaatgtccacctttggaccagtcccgggcaggcttgggcattttgccgccattgaatgatattgaatttaaatataaattcaagcatttgtgaatacatggctggaataagaggacactagatgatattacaggactgcagaacaccctcaccacacagtccctttggacaaatgcatttagtggtggtagaaatgattcaccaggtccaatgttgttcaccagtgcttgcttgtgaatcttaacattttggtgacagtttccagatgctatcacagactctgctagtgaaaagaactagtttctaggagcacaataatttgttttcatttgtataagtccatgaatgttcatatagccagggattgaagtttattattttcaaaggaaaacacctttattttattttttttcaaaatgaagatacacattacagccaggtgtggtagcaggcacctgtagtcttagctactcgagaggccaaagaaggaggatggcttgagcccaggagttcaagaccagcctggacagcttagtgagatcccgtctccgaagaaaagatatgtattctaattggcagattgttttttcctaaggaaactgctttatttttataaaactgcctgacaattatgaaaaaatgttcaaattcacgttctagtgaaactgcattatttgttgactagatggtggggttcttcgggtgtgatcatatatcataaaggatatttcaaatgattatgattagttatgtcttttaataaaaaggaaatatttttcaacttcttctatatccaaaattcagggctttaaacatgattatcttgatttcccaaaaacactaaaggtggttttattttcccttcatgttttaacttattgttgctgaaaactctatgtccggctttaactatcttctctatatttttatttcattcacattaatgagaagagttttctcagagattaaaaaaggtagtttttctgtcattgttaaatacacattatcactgaaaaaatgtagcttttatgtgatatgttttaaagttaaaactggatggaaatagccatttggaagctttggttatgaaacatgtggagtgtattaagtgcagcttgacattatgttttatttaaatgctttttatcgctaaatgacttgcagatgaaaaaaactaaggtgactcgagtgtttaaatgctgtgtacaacaatgctttgataaaatattttaagtatgagttatcagctctatgtcaattgatatttctgtgtagtatttatatttaaattatatttacctttttgcttattttacaaatattaagaaaatattctaacatttgataattttgaaatgattcatctttcagaaataaaagtatgaatct

SEQ ID NO: 6 - protein, 504 aa, Homo sapiens
MMTTSLIWGIAIAACCCLWLILGIRRRQTGEPPLENGLIPYLGCALQFGANPLEFLRANQRKHGHVFTCKLMGKYVHFITNPLSYHKVLCHGKYFDWKKFHFATSAKAFGHRSIDPMDGNTTENINDTFIKTLQGHALNSLTESMMENLQRIMRPPVSSNSKTAAWVTEGMYSFCYRVMFEAGYLTIFGRDLTRRDTQKAHILNNLDNFKQFDKVFPALVAGLPIHMFRTAHNAREKLAESLRHENLQKRESISELISLRMFLNDTLSTFDDLEKAKTHLVVLWASQANTIPATFWSLFQMIRNPEAMKAATEEVKRTLENAGQKVSLEGNPICLSQAELNDLPVLDSIIKESLRLSSASLNIRTAKEDFTLHLEDGSYNIRKDDIIALYPQLMHLDPEIYPDPLTFKYDRYLDENGKTKTTFYCNGLKLKYYYMPFGSGATICPGRLFAIHEIKQFLILMLSYFELELIEGQAKCPPLDQSRAGLGILPPLNDIEFKYKFKHL

SEQ ID NO: 7 - DNA, 1566 bp, Homo sapiens
  CDS (33)...(815)
cggagcgaggcagcgcgcccggctcccgcgccatggggcggctggtggctgtgggcttgctggggatcgcgctggcgctcctgggcgagaggcttctggcactcagaaatcgacttaaagcctccagagaagtagaatctgtagaccttccacactgccacctgattaaaggaattgaagctggctctgaagatattgacatacttcccaatggtctggctttttttagtgtgggtctaaaattcccaggactccacagctttgcaccagataagcctggaggaatactaatgatggatctaaaagaagaaaaaccaagggcacgggaattaagaatcagtcgtgggtttgatttggcctcattcaatccacatggcatcagcactttcatagacaacgatgacacagtttatctctttgttgtaaaccacccagaattcaagaatacagtggaaatttttaaatttgaagaagcagaaaattctctgttgcatctgaaaacagtcaaacatgagcttcttccaagtgtgaatgacatcacagctgttggaccggcacatttctatgccacaaatgaccactacttctctgatcctttcttaaagtatttagaaacatacttgaacttacactgggcaaatgttgtttactacagtccaaatgaagttaaagtggtagcagaaggatttgattcagcaaatgggatcaatatttcacctgatgataagtatatctatgttgctgacatattggctcatgaaattcatgttttggaaaaacacactaatatgaatttaactcagttgaagggtacttgagctggatacactggtggataatttatctattgatccttcctcgggggacatctgggtaggctgtcatcctaatggccagaagctcttcgtgtatgacccgaacaatcctccctcgtcagaggttctccgcatccagaacattctatctgagaagcctacagtgactacagtttatgccaacaatgggtctgttctccaaggaagttctgtagcctcagtgtatgatgggaagctgctcataggcactttataccacagagccttgtattgtgaactctaaattgtacttttggcatgaaagtgcgataacttaacaattaattttctatgaattgctaattctgagggaatttaaccagcaacattgacccagaaatgtatggcatgtgtagttaattttattccagtaaggaacggcccttttagttcttagagcacttttaacaaaaaaggaaaatgaacaggttctttaaaatgccaagcaagggacagaaaagaaagctgctttcgaataaagtgaatacattttgcacaaagtaagcctcacctttgccttccaactgccagaacatggattccactgaaatagagtgaattatatttccttaaaatgtgagtgacctcacttctggcactgtgactactatggctgtttagaactactgataacgtattttgatgttttgtacttacatctttgtttaccattaaaaagttggagttatattaaagactaactaaaatccca

SEQ ID NO: 8 - protein, 261 aa, Homo sapiens
MGRLVAVGLLGIALALLGERLLALRNRLKASREVESVDLPHCHLIKGIEAGSEDIDILPNGLAFFSVGLKFPGLHSFAPDKPGGILMMDLKEEKPRARELRISRGFDLASFNPHGISTFIDNDDTVYLFVVNHPEFKNTVEIFKFEEAENSLLHLKTVKHELLPSVNDITAVGPAHFYATNDHYFSDPFLKYLETYLNLHWANVVYYSPNEVKVVAEGFDSANGINISPDDKYIYVADILAHEIHVLEKHTNMNLTQLKGT

SEQ ID NO: 9 - DNA, 897 bp, Homo sapiens
  CDS (39)...(839)
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatttggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 10 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 11 - DNA, 1121 bp, Mus musculus
  CDS (1)...(1062)
  misc_feature (1)...(1121): n = A,T,C or G
atggggcacctcgtggcgctgcccttgctgggagcctgtctggccttaatangggnaaggctgctgaattttagagaacgagttagtacaactcgagaaataaaggccacagaaccacaaaactgccacctgattgagggcctcgagaatggctctgaagatattgatatacttcctagcgggctggcttttatctccactggattaaaatatccgggcatgccagcgtttgcaccggacaaaccaggaagaatctttctgatggatctgaatgagcaaaacccagaggcgcaagcactggaaatcagtggtgggcttgaccaggagtcactaaatcctcacgggatcagcactttcatcgacaaagacaacactgcttatctttatgtcgtgaatcaccccaacatggactccactgtggagatatttaagtttgaagaacaacaacgctctctcatccacctgaaaactctaaaacatgaacttctcaagagtgtgaatgacattgtggttcttgggccagagcagttctatgccacaagagaccattactttaccagttatttcttggtacttctggagatgatcttggaccctcactggacttccgtcgttttctacagcccaaaagaggtcaaagttgtggcccaaggattcagttctgccaacggaatcacagtctcactagaccagaagtttgtctatgtagctgatgtaacagctaagaacattcacataatggaaaaacatgataattgggatttaactccagtgaaggtcattcagctggggaccttagtggataacctgaccgttgctccagccacgggagatattttggcaggctgccaccctaaccccatgaagctgttgatctataatcctgagggccctccaggatcagaagtactacgcatccaggactctttgtcagataagcccagggtgagcacactgtatgcgaacaacggctctgtgcttcagggcagcaccgtggcttctgtgtatcataagagaatgctcataggtactatatttcacaaagctctgtactgtgacctctagatctttctaaaacggtctttatatttggcaaaagtaaaattgtaatttgtatgcta

SEQ ID NO: 12 - protein, 354 aa, Mus musculus
  VARIANT (1)...(354): Xaa = Any Amino Acid
MGHLVALPLLGACLALIXXRLLNFRERVSTTREIKATEPQNCHLIEGLENGSEDIDILPSGLAFISTGLKYPGMPAFAPDKPGRIFLMDLNEQNPEAQALEISGGLDQESLNPHGISTFIDKDNTAYLYVVNHPNMDSTVEIFKFEEQQRSLIHLKTLKHELLKSVNDIVVLGPEQFYATRDHYFTSYFLVLLEMILDPHWTSVVFYSPKEVKVVAQGFSSANGITVSLDQKFVYVADVTAKNIHIMEKHDNWDLTPVKVIQLGTLVDNLTVAPATGDILAGCHPNPMKLLIYNPEGPPGSEVLRIQDSLSDKPRVSTLYANNGSVLQGSTVASVYHKRMLIGTIFHKALYCDL

SEQ ID NO: 13 - DNA, 2382 bp, Homo sapiens
cactcgagaaatttacttaccatctctaaacattaatttctcaattagggaagtgatatgacaaagttgctgtgaagtcaaatgaaggaatgaatgcaaaatcccaaccctttacatattacctctgttctcaattaatcattggcttgaactgaacatttttaacatggttccctgctaaaggtgtttttacatatagtaagacctaatagataatattatgttacataatctataattattatatataaaactatataaaattatatagtacctatttagacatataaaaagggaagttttttaatgaaaaattagattggctgaagagtgtggaaatcatcacatagggaagtggtcagcactgtggatatacaccaaacagtattacagagacatactgaaaataaagcacactgacagaaagaaacctcattaggatagaattttgggagattggtgtcttgttattcccagagtctgacttttctaatcacgtagcattgtcagggatatctaaattttaaacttcagattttgtacaaaaaaacctaaaaggactatattttaaaataagaagcatgtacaaataattgtaaaggaaagagtgtgagttttgtagtctccagttctgtgtcagtaagaatttgatgaggaatagccaatagcatttatgctggtccaggctggatcaaattgtgatcgtacttctagtcttcaagctgcctggttagaaattatgcagcttactttgatgcctgtcctactactactattagccttttagtggttaccttaagaataactgaataatactgttactttgcatctctcaaagaccgtgcgctgttcccgcatactttcttgcagacgtattgtcttctgaaggatggtgcccatgggcacagcaatttggggcatcttttggtaggaagcatgatttctcctgtttgggcacaaaagagtgactacggaatataagctaaacaccttctcccagaaatatcagttgtaaaacttagctcattaatgtaatgttgaacgttttcatgtgagtcatacttgaacttcttgaatgtgagtaaataacggagttgaattgtataaaaccctgatggatggcagaagggaaagaacatccctttcatctgatccatgcctgagattccttccctctactatactactggaaataaacaacttgtctggtgctctaaaaattaatgctattatcattatagagagaacaggagtgagtgcttttaggttattacttgttcttactaaggcatcatatctttcttcacgtgttttgtgtttgaggctaagggataaaggtacaggaaaaaggatctaagaaaaaaactcgaatctctaaacatggagtctttttttttttttttttttttgaggtggagtctcactctgttgcccaggctggagtgcagtggtgcgatctctgctcactgcaacctccgcctcctgggttcaagcaattcttcagcctcagcctcccgagtagctgggattacagcgcatgcctggccaatttttgtatttttagtagagactagtttcaccatgttggccaggctgttctagagctcctagcctcaagtgatccagccacctcggccacccaaagtgctgggattacaggcatgagccaccacacctagccaaacatggagtctttactcaacattatagtaacgtgaaagaggagatcatgagaattaatgtatgttttagaaggcatagattacttataaaaaaggaaagatcaggctgggcacggtggctcgcgcctgtaatcccagcactttgggaggccaaggcgagcggaccatgaggtcaggggatcaagaccatcctgaccaacatggcgaaaccctgtctctactaaaatacaaaaaattagccgggtgtgatgcacacgcctgtagaacctgggaggcagaggttgcagtgagctgagatcacaccactgcactccaccctggtgagacagcgagactccatctccaaaaaaaaaggaaagctcaatctgctgtaaattatgtgcttgtttcaacaacccttgtttcttttccttttcacttctcttttttttttaaagcggcctaaatggggtgaagagtgagttatctgacaaatttagattttgcaaacctgtgcattgatgagagtgctattgaaacacattaagaaagattttcaacgcaggaatgtgtcatttcctttcttcatgtaccagatgctgaaatactatgagataaagattttaggtttcaattgtaaagagagagaagtggataaatcagtgctgctttctttaggacgaaaggtaaagaaaaaaaaaggcttattttaatgtttttt

(19) United States
(12) Patent Application Publication
(10) Pub. No.: US 2004/0001810 A1
(43) Pub. Date: Jan. 1, 2004

(54) Compositions and methods for treating atherosclerosis

(76) Inventor: Roger A. Davis, Solana Beach, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/186,288

(22) Filed: Jun. 26, 2002

(51) Int. Cl. C07H 21/04; A61K 48/00; A01K 67/027; C12N 9/02; C12P 21/02; C12N 5/08

(52) U.S. Cl. 424/93.21; 435/69.1; 435/189; 435/320.1; 435/325; 435/366; 536/23.2; 800/14

(57) ABSTRACT

The invention provides a nucleic acid having a nucleotide sequence encoding an inhibitor of a pro-atherogenic molecule operationally linked to a macrophage-specific expression element. The invention also provides a recombinant macrophage expressing a nucleic acid encoding an inhibitor of a pro-atherogenic molecule. The invention further provides a method for inhibiting or reducing atherosclerosis including administering to an individual a population of recombinant cells expressing a nucleic acid encoding an inhibitor of a pro-atherogenic molecule. Additionally, the invention provides a method for inhibiting or reducing atherosclerosis including administering to an individual a nucleic acid encoding an inhibitor of a pro-atherogenic molecule, the inhibitor of a pro-atherogenic molecule operationally linked to a macrophage-specific expression element.

This application claims benefit of the filing date of U.S. Provisional Application No. 60/______ filed Jun. 26, 2001, which was converted from U.S. Ser. No. 09/893,366, and which is incorporated herein by reference.

This invention was made with government support under grant number 1RO1 HL 57974 awarded by National Institute of Health. The United States Government has certain rights in this invention.

### BACKGROUND OF THE INVENTION

The present invention relates generally to the field of cardiovascular medicine and more specifically to atherosclerosis.

Atherosclerotic heart disease and stroke are the number one cause of death in the developed world. Atherosclerosis is a disease of the arteries, which are blood vessels that carry blood from the heart to the rest of the body. Atherosclerosis is characterized by an accumulation along vessel walls of fatty deposits, or plaques. These plaques narrow the vessel diameter, resulting in reduced blood flow, and can harden, causing the vessel wall to become brittle. Large plaques can be unstable and are prone to rupture, causing the release of particles that can occlude vessels downstream. In addition, factors produced at the plaque surface can stimulate the formation of blood clots, which can occlude blood flow at the plaque site or at a smaller distal vessel. Heart attack can be caused by blockage of a coronary artery that supplies blood to the heart muscle, while stroke can result from blockage of a carotid or vertebral artery that supplies blood to the brain. Thus, the effects of atherosclerosis in an individual can be many and severe.

One of the most important risk factors associated with atherosclerotic heart disease is the concentration in the blood of low density lipoprotein (LDL), or “bad cholesterol.” High levels of LDL can be caused by genetically programmed increased liver production of LDL or decreased clearance of LDL from the bloodstream, increased dietary intake of cholesterol, obesity, and most commonly, a combination of these factors. An individual having an elevated LDL cholesterol level can be treated with medication to both prevent and decrease progression of atherosclerosis. Four major classes of drugs are commonly used to treat high cholesterol levels, including HMG CoA reductase inhibitors that slow cholesterol production (often referred to as statins), bile acid sequestrants (cholestyramine and colestipol) that prevent recycling of bile acids, nicotinic acid (Niacin) and fibrates. These drugs have various shortcomings including lack of specificity, lack of efficacy and adverse side effect profiles. In addition, considerable variation in the magnitude of LDL-cholesterol response to drug therapy exists in individual patients.

Thus, there exists a need for therapeutic agents to treat atherosclerosis and methods for identifying therapeutic agents for treatment of atherosclerosis. The present invention satisfies this need and provides related advantages as well.

### SUMMARY OF THE INVENTION

The invention provides a nucleic acid having a nucleotide sequence encoding an inhibitor of a pro-atherogenic molecule operationally linked to a macrophage-specific expression element. The invention also provides a recombinant macrophage expressing a nucleic acid encoding an inhibitor of a pro-atherogenic molecule. The invention further provides a method for inhibiting or reducing atherosclerosis including administering to an individual a population of recombinant cells expressing a nucleic acid encoding an inhibitor of a pro-atherogenic molecule. Additionally, the invention provides a method for inhibiting or reducing atherosclerosis including administering to an individual a nucleic acid encoding an inhibitor of a pro-atherogenic molecule, the inhibitor of a pro-atherogenic molecule operationally linked to a macrophage-specific expression element.

### BRIEF DESCRIPTION OF THE DRAWINGS

FIG. 1 shows expression of CYP7A1 mRNA in the tissues of 7aMac transgenic mice.

FIG. 2 shows expression of PON1 mRNA in the tissues of transgenic mice.

FIG. 3 shows PON1 enzyme activity in plasma of non-transgenic and PON1 transgenic mice.

FIG. 4 shows expression of CYP7A1 mRNA in recipient mice receiving bone marrow cells derived from CYP7A1 transgenic mice.

FIG. 5 shows expression of the CYP7A1 transgene in bone marrow recipient LDL receptor −/− mice.

FIG. 6 shows reduction in the formation of atherosclerosis in LDL receptor −/− mice transplanted with bone marrow from CYP7A1 transgenic mice after being fed a cholesterol-rich diet.

FIG. 7 shows expression of PON1 mRNA in circulating white blood cells obtained from LDL receptor mice transplanted with bone marrow derived from PON1 transgenic mice and non-transgenic littermates.

FIG. 8 shows reduction in the formation of atherosclerosis in LDL receptor −-− mice transplanted with bone marrow from PON1 transgenic mice after being fed a cholesterol-rich diet.

FIG. 9 shows the nucleotide sequence of a human class A scavenger receptor enhancer including the sequence from about −4.1 to about −4.5 kb from the major transcription start site of the human class A scavenger receptor gene.

### DETAILED DESCRIPTION OF THE INVENTION

The present invention provides methods for inhibiting or reducing atherosclerosis by recombinant expression of an inhibitor of a pro-atherogenic molecule. The invention also provides a nucleic acid having a nucleotide sequence encoding an inhibitor of a pro-atherogenic molecule operationally linked to a macrophage-specific expression element. An inhibitor of a pro-atherogenic molecule is capable of preventing formation of foam cells as well as smooth muscle cell growth. The invention includes a variety of inhibitors of pro-atherogenic molecules including, for example, a paraoxonase polypeptide, apolipoprotein A1 polypeptide, or cholesterol-7α-hydroxylase polypeptide. A nucleic acid of the invention can be used for targeted expression of an inhibitor of a pro-atherogenic molecule in a macrophage. An advantage of targeted expression of inhibitor of a pro-atherogenic molecule in a macrophage is that the inhibitor is localized to the site of plaque formation since macrophages are commonly recruited during inflammatory responses leading to plaque formation and eventually become incorporated into a formed plaque as foam cells.

The methods of the invention can be used to treat an individual suffering from, or at risk for developing, atherosclerosis. Accordingly, the invention provides a method for inhibiting or reducing atherosclerosis using macrophage-specific expression of an inhibitor of a pro-atherogenic molecule.

The invention further provides a recombinant cell expressing an inhibitor of a pro-atherogenic molecule. A recombinant cell of the invention that expresses an inhibitor of a pro-atherogenic molecule can be used to treat an individual having or at risk for developing atherosclerosis. Thus, the invention provides a method for inhibiting or reducing atherosclerosis by administering to an individual a population of recombinant cells expressing an inhibitor of a pro-atherogenic molecule. A recombinant cell expressing an inhibitor of a pro-atherogenic molecule can be a macrophage. A recombinant macrophage expressing an inhibitor of a pro-atherogenic molecule and administered to an individual in a method of the invention provides the advantage of directing the gene product to the site of atherogenesis or atherosclerosis. Further specificity can be achieved by operationally linking a nucleic acid encoding an inhibitor of a pro-atherogenic molecule to a macrophage-specific expression element. However, expression of an inhibitor of a pro-atherogenic molecule in a recombinant macrophage of the invention can be under the control of any expression element that is active in the macrophage including, for example, a non-tissue specific promoter, or inducible promoter. In such a case, macrophage-specific expression can be achieved by transfecting a nucleic acid encoding an inhibitor of a pro-atherogenic molecule into a macrophage in vitro and administering the recombinant macrophage to an individual, thereby providing macrophage-specific expression without the need to use a macrophage specific expression element. Thus, a recombinant cell of the invention can provide advantages particular to the promoter, such as those described below, while maintaining the specificity provided by macrophage localization.

A recombinant cell of the invention can also be used to screen for a drug potentially effective for treating atherosclerosis. Accordingly, the invention provides a method of identifying a compound that reduces susceptibility to developing atherosclerosis. The method can be used to identify a compound that affects an activity of an inhibitor of a pro-atherogenic molecule such as interaction with a pro-atherogenic molecule, modification of a pro-atherogenic molecule, production of a secondary molecule that affects activity of a pro-atherogenic molecule, or depletion of a secondary molecule that affects activity of a pro-atherogenic molecule.

The invention further provides a transgenic non-human animal having recombinant cells expressing an inhibitor of a pro-atherogenic molecule. A transgenic non-human animal can be used in a drug screening method similar to that described above for a recombinant cell of the invention. A transgenic animal of the invention can be advantageous for determining both the effects of a candidate compound on the activity of an inhibitor of a pro-atherogenic molecule and on atherosclerotic plaque formation in vivo. Therefore, the invention provides a method for identifying a compound that reduces susceptibility to developing atherosclerosis. The method can include the steps of (a) contacting a cell expressing an inhibitor of a pro-atherogenic molecule with a candidate compound and a pro-atherogenic molecule; (b) determining an activity of the pro-atherogenic molecule in the presence of the inhibitor of a pro-atherogenic molecule and the candidate compound; and (c) identifying a compound that increases the inhibitory effects of the inhibitor toward the pro-atherogenic molecule, the compound being a compound that reduces susceptibility to eveloping atherosclerosis.

As used herein the term “inhibitor of a pro-atherogenic molecule” is intended to mean a molecule that, when in the presence of a pro-atherogenic molecule, reduces an activity of the pro-atherogenic molecule that is associated with the initiation or progression of atherosclerosis. An inhibitor that reduces an activity of a pro-atherogenic molecule can be any type of molecule that reduces pro-atherogenic activity. For example, an inhibitor of a pro-atherogenic molecule can be a gene product such as a polypeptide or protein, a nucleic acid such as a DNA or RNA, or a molecule produced or modified by a gene product. The inhibitor molecule can be, for example, a polypeptide having paraoxonase activity such as a PON1 gene product, or functional fragment thereof; polypeptide having cholesterol-7α-hydroxylase activity such as a CYP7A1 gene product, or functional fragment thereof; or polypeptide having apolipoprotein A1 activity such as an APOA1 gene product, or functional fragment thereof. The inhibitor molecule can reduce an activity of the pro-atherogenic molecule by binding to the pro-atherogenic molecule, producing a molecule that reduces activity of the pro-atherogenic molecule, or depleting a molecule that increases activity of the pro-atherogenic molecule.

As used herein the term “nucleic acid” is intended to mean a polymer of nucleotide units. The term can include naturally occurring polymers such as polydeoxyribonucleic acid (DNA) and polyribonucleic acid (RNA) and analogs thereof. Examples of naturally occurring DNA include genomic DNA (gDNA), copy DNA (cDNA) and extragenomic DNA such as non-chromosomal plasmids and vectors. Naturally occurring RNA can be, for example, messenger RNA (mRNA). The term can also include an analog of a naturally occurring polymer of nucleotide units so long as the polymer can encode a PON1 polypeptide or an expression element. A polymer included in the term is understood to contain any number of nucleotides greater than 2 and can be double stranded or single stranded. When expressed in a transgenic animal, a DNA encoding a polypeptide can be referred to as a transgene.

As used herein the term “polypeptide” is intended to mean a polymer of 2 or more amino acids connected by one or more peptide bond.

As used herein the term “paraoxonase,” when used in reference to a polypeptide, is intended to mean a polypeptide having esterase activity. The term can include broad specificity esterase activity characterized by the ability to hydrolyze esters in a wide variety of substrates or specificity for a particular substrate. Substrates that can be hydrolyzed by a polypeptide having esterase activity include, for example, diisopropylfluorophosphate, soman, sarin, 4-nitro-phenylacetate, 2-nitro-phenylacetate, 2-naphthylacetate, or phenylthioacetate as described in Smolen et al., Drug Metab. Dispos. 19:107-112 (1991), oxodized LDL or chloropyrifos oxon as described in Shih et al., Nature 394:284-287 (1998) or phospholipid and cholesteryl ester hydroperoxides derived from arachidinic and linoleic acid as described in Mackness et al., Curr. Opin. Lipid. 11:383-388 (2000). The term can include a polypeptide additionally having phospholipase activity.

A paraoxonase can be a “PON1” polypeptide having a sequence identical to or substantially the same as SEQ ID NO: 2, a “PON2” polypeptide having a sequence identical to or substantially the same as SEQ ID NO: 8, or a “PON3” polypeptide having a sequence identical to or substantially the same as SEQ ID NO: 12. It is understood that minor modifications can be made without destroying PON1, PON2 or PON3 polypeptide activity and that only a portion of the primary structure can be required in order to effect activity. Such modifications are included within the meaning of the terms so long as the modified polypeptide has at least one PON1, PON2 or PON3 polypeptide activity, respectively that is sufficient for inhibiting activity of a pro-atherogenic molecule. It is understood in the art that an activity of a polypeptide can include specificity of binding to a particular reactant, the nature of the chemical reaction catalyzed, or the rates at which substrates are associated, dissociated, or chemically converted to product, as well as the rate at which product is released. Minor modifications included in the terms and methods for identifying minor modifications and substantially similar polypeptides are described below.

As used herein the term “cholesterol-7α-hydroxylase,” when used in reference to a polypeptide, is intended to mean a polypeptide having an activity capable of converting cholesterol to 7α-hydroxycholesterol. The term can include a product of the CYP7A1 gene, or functional fragment thereof. Various mammalian CYP7A1 nucleotide and amino acid sequences are publically available, for example, in the GenBank data base. A rat CYP7A1 sequence (SEQ ID NOS: 3 and 4 for nucleotide and amino acid sequences, respectively) is available at Genbank accession No. J05430. The protein product of this rat CYP7A1 gene is composed of 503 amino acid residues with a calculated molecular weight of 16.6 kDa. Additionally, a human CYP7A1 sequence (SEQ ID NOS: 5 and 6 for nucleotide and amino acid sequences, respectively) is available at Genbank accession No. XM_{—}005022.

As used herein the term “apolipoprotein A1,” when used in reference to a polypeptide, is intended to mean a polypeptide having a structural role in a High Density Lipoprotein particle and acting as a cofactor or activator of lecithin-cholesterol-acetyltransferase (LCAT). The term is intended to be consistent with its use in the art as described, for example, in Bennett and Plum, CECIL Textbook of Medicine, 20^{th} Ed., W. B. Saunders Co., Philadelphia (1996). The term can include a product of the APOA1 gene, or functional fragment thereof. A human apolipoprotein (APOAL) sequence (SEQ ID NOS: 9 and 10 for nucleotide and amino acid sequences, respectively) is available at Genbank accession No. XM_{—}006435. The protein product of the human APOA1 gene is composed of 267 amino acid residues.

As used herein the term “expression element” is intended to mean a nucleic acid sequence that regulates transcription or translation of a nucleic acid sequence. The term can include constitutive or inducible regulation of transcription or translation. The term can also include tissue or cell specific regulatory sequences. Examples of sequences that regulate transcription include, for example, promoters, enhancers, silencers and the like. Examples of sequences that regulate translation include, for example, internal ribosome entry sites, or response elements. Accordingly, the term “regulate,” or grammatical derivatives thereof, when used in reference to a nucleic acid encoding a polypeptide, are intended to refer to control of nucleic acid or polypeptide expression in a constitutive, suppressible or inducible manner.

As used herein, the term “macrophage-specific expression” is intended to mean transcription or translation of a nucleic acid in a macrophage. The term can include transcription or translation of a nucleic acid under the control of any expression element that is active in a macrophage including, for example, under the control of a tissue-specific expression element, constitutive expression element, or inducible expression element. Thus, the term can include transcription or translation under the control of an expression element that is active in one or more cell types, so long as expression occurs in a macrophage. Macrophage-specific expression can also occur when a macrophage is genetically modified in vitro to express an inhibitor of a pro-atherogenic molecule resulting in expression of a transgene in the macrophage.

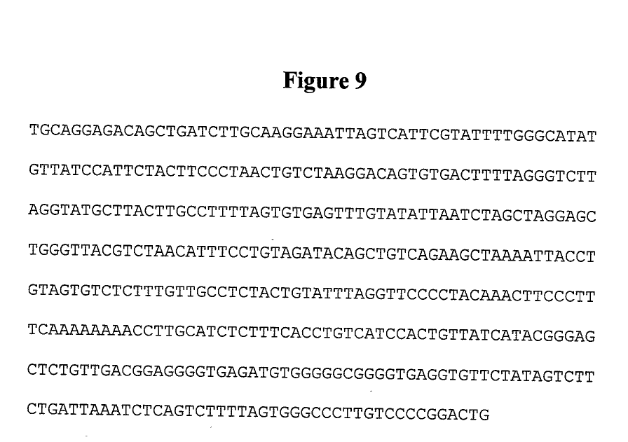

As used herein the term “macrophage-specific expression element” is intended to mean a nucleic acid sequence that activates transcription or translation of a nucleic acid in a macrophage. The term can also include an expression element that represses expression in a non-macrophage cell. The term can include a class A scavenger receptor expression element described in Horvai et al., Proc. Natl. Acad. Sci. USA 92:5391-5395 (1995), Moulton et al., Mol. Cell. Biol. 14:4408-4418 (1994), Moulton et al., Proc. Natl. Acad. Sci. USA 89:8102-8106 (1992) and Wu et al., Mol. Cell. Biol. 14:2129-2139 (1994) including, for example, human class A scavenger receptor expression elements provided in SEQ ID NO: 13 (GenBank accession No. M93189) or shown in FIG. 9. For example, a scavenger receptor expression element can include a class A scavenger receptor promoter sequence extending from about −696 to about +46 base pairs from the major transcription start site of the SR gene; a class A scavenger receptor core promoter, which can include a sequence extending from about −245 to about +46 base pairs from the major transcription start site of the SR gene or a class A scavenger receptor enhancer, which can include sequences from about −4.1 to about −4.5 kb from the major transcription start site.

As used herein the term “operationally linked,” when used in reference to an expression element and an expressed nucleic acid sequence is intended to mean connected in an orientation that allows the expression element to regulate expression of the nucleic acid sequence. An expression element can be operationally linked in an orientation upstream or downstream of an expressed sequence or the transcription start site.

As used herein the term “recombinant,” when used in reference to a cell or nucleic acid, is intended to mean containing a nucleic acid sequence that is non-naturally occurring in the cell or nucleic acid, containing a naturally occurring nucleic acid sequence in a non-natural location or in multiple copies in a natural location where such multiple copies do not naturally occur. A non-naturally occurring sequence included in the term can be an expression element, or polypeptide coding sequence. A non-natural location can include a location in a genomic DNA such as a chromosome or an extrachromosomal location such as a plasmid. In a cell the nucleic acid sequence can be expressed stably or transiently.

As used herein the term “embryonic stem cell” is intended to mean a pluripotent cell type derived from an embryo which can differentiate to give rise to all cellular lineages. Thus, an ES cell can differentiate to a neuronal cell, hematopoietic cell, muscle cell, adipose cell, germ cell or any other cellular lineage. Examples of cell markers that indicate a human embryonic stem cell include the Oct-4 transcription factor, alkaline phosphatase, SSEA-4, TRA 1-60, and GCTM-2 epitope as described in Reubinoff et al., Nat. Biotech. 18:399-404 (2000).

As used herein the term “isolated” as a modifier of nucleic acid or polypeptide is intended to mean that the nucleic acid or polypeptide so designated has been produced in such form by the hand of man, and thus is separated from its native environment.

As used herein the term “transgenic,” when used in reference to an organism, is intended to mean containing a stably incorporated nucleic acid sequence that is non-naturally occurring in the organism or incorporated at a non-natural location of the organism's genome such that the nucleic acid sequence can be passed on to progeny. Accordingly, a nucleic acid sequence present in an organism that is non-naturally occurring in the organism or incorporated at a non-natural location of the organism's genome is referred to herein as a “transgenic nucleic acid.”

As used herein the term “atherosclerosis” is intended to mean a form of arteriosclerosis characterized by formation of a plaque. Early lesions of a plaque can be characterized as a fatty streak consisting of lipid-laden foam cells which are macrophages that have migrated as monocytes into the subendothelial layer of the intima. The plaque can form a fibrous plaque consisting of intracellular and extracellular lipids, smooth muscle cells, connective tissue and glycosaminoglycans. Symptoms indicative of atherosclerosis are described, for example, in The Merck Manual, Sixteenth Ed, (Berkow, R., Editor) Rahway, N.J., (1992) and Bennett and Plum, supra (1996) and can include, for example, reduced systolic expansion, abnormally rapid wave propagation, reduced elasticity of the affected arteries, angina, intermittent claudication, critical stenosis, thrombosis, aneurysm, or embolism.

As used herein the term “reduced susceptibility,” when used in reference to a disease or condition is intended to mean having a lower probability or potential of being affected by the disease or condition. Being affected by a disease or condition can include displaying a symptom, diagnostic marker or characteristic of the disease or condition. The term can refer to the probability or potential of an unaffected individual becoming affected or of an affected individual becoming increasingly affected. A lower probability of being affected by a disease or condition can be determined relative to another individual or population. A lower probability of being affected by a disease or condition can also be determined relative to self prior to, or after a particular treatment. A lower potential of being affected by a disease or condition can include decreased risk factors, decreased quantity or activity of a disease associated factor, or increased quantity or activity of a factor that reverses or prevents the disease or condition, or symptom thereof. For example, the methods of the invention can be used to reduce formation or persistence of fatty streaks at the subendothelial layer of the intima or to reduce deposition of intracellular and extracellular lipids, smooth muscle cells, connective tissue or glycosaminoglycans in an artery, thereby reducing susceptibility to atherosclerosis.

As used herein the term “inhibiting,” when used in reference to a disease or condition, is intended to mean preventing or forestalling occurrence of the disease or condition, or symptom thereof. The term can include the prophylactic treatment of an individual to guard from the occurrence of a disease or condition. The term can also include arresting the development or progression of the disease or condition. When used in reference to atherosclerosis, the term can include preventing or forestalling plaque formation, reduced systolic expansion, abnormally rapid wave propagation, or reduced elasticity of the affected arteries. The term can also include, for example, inhibiting or arresting the progression of one or more pathological conditions or chronic complications associated with the disease or condition such as, in the case of atherosclerosis, angina, intermittent claudication, critical stenosis, thrombosis, aneurysm, or embolism.

As used herein the term “reducing,” when used in reference to a disease or condition, is intended to mean lessening the extent or a symptom of the disease or condition. The term can include reversing the development or progression of a disease or condition or symptom thereof. When used in reference to atherosclerosis, the term can include lessening plaque size, increasing systolic expansion, normalizing wave propagation, or increasing elasticity of affected arteries. The term can also include, for example, lessening one or more pathological conditions or chronic complications associated with the disease or condition such as, in the case of atherosclerosis, angina, intermittent claudication, critical stenosis, thrombosis, aneurysm, or embolism.

The invention provides a nucleic acid having a nucleotide sequence encoding an inhibitor of a pro-atherogenic molecule operationally linked to a macrophage-specific expression element. Nucleic acids encoding inhibitors of pro-atherogenic molecules are known in the art, as described herein, and can be obtained by known cloning methods including, for example, isolation from a cDNA library or genomic library with a natural or artificially designed gene-specific nucleic acid probe. Another useful method for producing a nucleic acid encoding an inhibitor of a pro-atherogenic molecule involves amplification of the nucleic acid molecule using PCR and a sequence specific nucleic acid probe. These and other cloning methods are well known in the art as described, for example, in Sambrook et al., Molecular Cloning: A Laboratory Manual, 2nd ed., Cold Spring Harbor Press, Plainview, N.Y. (1989); Sambrook et al., Molecular Cloning: A Laboratory Manual, 3rd ed., Cold Spring Harbor Press, Plainview, N.Y. (2001); Ausubel et al. (Current Protocols in Molecular Biology (Supplement 47), John Wiley & Sons, New York (1999)).

A macrophage-specific expression element included in a nucleic acid of the invention can be a macrophage-specific promoter such as a class A scavenger receptor promoter. A nucleic acid of the invention can include a sequence of a macrophage-specific enhancer such as a class A scavenger receptor enhancer. The expression elements can be used individually or in various combinations to suit a particular application of the methods. Class A scavenger receptor expression elements prevent expression of an operationally attached gene in macrophage precursor cells such as monocytes and activate expression of the gene upon macrophage differentiation as described in Horvai et al., supra (1995). Class A scavenger receptor expression elements induce expression in the presence of macrophage colony-stimulating factor (M-CSF), granulocyte macrophage colony-stimulating factor (GM-CSF), and phorbol ester phorbol 12-myristate 13-acetate (PMA). A macrophage-specific expression element can be operationally linked to a sequence encoding an inhibitor of a pro-atherogenic molecule according to known properties and orientations of the expression element. Cloning methods useful for linking two nucleic acid sequences are known in the art as described, for example, in Sambrook et al., supra (1989); Sambrook et al., supra (2001) and Ausubel et al., supra (1999)).

A nucleic acid molecule of the invention can include the nucleotide sequence of an inhibitor of a pro-atherogenic molecule such as any paraoxonase polypeptide including, for example, gene products of PON1 (Li et al., Pharmacogenomics 7:137-144 (1997)), PON2 (Mochizuki et al., Gene 213:149-157 (1998)) or PON3 (Reddy et al., Arterioscler. Thromb. Vasc. Biol. 21:542-547 (2001) and Draganov et al., J. Biol. Chem. 275:33435-33442 (2000)). For example, a nucleic acid molecule of the invention can include the sequence of the human PON1 cDNA, referenced as SEQ ID NO: 1 (GenBank accession No. XM_{—}004948), or a fragment thereof. A nucleic acid encoding a PON1 polypeptide includes sequences that are the same or substantially the same as SEQ ID NO: 1. Other nucleic acid molecules encoding paraoxonase polypeptides useful in the invention include, for example, the sequence of the human PON2 cDNA, referenced as SEQ ID NO: 7 (GenBank accession No. XM_{—}004947), the sequence of the mouse PON3 cDNA, referenced as SEQ ID NO: 11 (GenBank accession No. NM_{—}008897), or a fragment thereof. A nucleic acid encoding a PON1, PON2 or PON3 polypeptide includes a sequence that is the same or substantially the same as SEQ ID NO: 1, SEQ ID NO: 7 or SEQ ID NO: 11, respectively.

A nucleic acid sequence that is substantially the same as a reference sequence includes one that encodes the same polypeptide amino acid sequence. Such sequences are commonly referred to in the art as having silent differences due to degeneracy of the genetic code.

Methods for determining that two sequences are substantially the same are well known in the art. For example, one method for determining if two sequences are substantially the same is BLAST, Basic Local Alignment Search Tool, which can be used according to default parameters as described by Tatiana et al., FEMS Microbial Lett. 174:247-250 (1999) or on the National Center for Biotechnology Information web page at ncbi.nlm.gov/BLAST/. BLAST is a set of similarity search programs designed to examine all available sequence databases and can function to search for similarities in amino acid or nucleic acid sequences. A BLAST search provides search scores that have a well-defined statistical interpretation. Furthermore, BLAST uses a heuristic algorithm that seeks local alignments and is therefore able to detect relationships among sequences which share only isolated regions of similarity including, for example, protein domains (Altschul et al., J. Mol. Biol. 215:403-410 (1990)).

In addition to the originally described BLAST (Altschul et al., supra, 1990), modifications to the algorithm have been made (Altschul et al., Nucleic Acids Res. 25:3389-3402 (1997)). One modification is Gapped BLAST, which allows gaps, either insertions or deletions, to be introduced into alignments. Allowing gaps in alignments tends to reflect biologic relationships more closely. For example, gapped BLAST can be used to identify sequence identity within similar domains of two or more polypeptides. A second modification is PSI-BLAST, which is a sensitive way to search for sequence homologs. PSI-BLAST performs an initial Gapped BLAST search and uses information from any significant alignments to construct a position-specific score matrix, which replaces the query sequence for the next round of database searching. A PSI-BLAST search is often more sensitive to weak but biologically relevant sequence similarities.

A second resource that can be used to determine if two sequences are substantially the same is PROSITE, available on the world wide web at ExPASy. PROSITE is a method of determining the function of uncharacterized polypeptides translated from genomic or cDNA sequences (Bairoch et al., Nucleic Acids Res. 25:217-221 (1997)). PROSITE consists of a database of biologically significant sites and patterns that can be used to identify which known family of polypeptides, if any, the new sequence belongs. Using this or a similar algorithm, a polypeptide that is substantially the same as another polypeptide can be identified by the occurrence in its sequence of a particular cluster of amino acid residues, which can be called a pattern, motif, signature or fingerprint, that is substantially the same as a particular cluster of amino acid residues in a reference polypeptide including, for example, those found in similar domains. PROSITE uses a computer algorithm to search for motifs that identify polypeptides as family members. PROSITE also maintains a compilation of previously identified motifs, which can be used to determine if a newly identified polypeptide is a member of a known family.

Sequence comparison can include a full sequence of a gene, cDNA or expressed products thereof or can include one or more particular regions thereof. A particular region can be identified by visual inspection of a sequence alignment to identify regions of relatively high homology or similarity. Those regions can be crossreferenced with structural data to find correlations between a particular structural domain and region of homology. A structural model of a reference polypeptide such as a PON1, CYP7A1 or APOAL gene product can also be used in an algorithm that compares polypeptide structure including, for example, SCOP, CATH, or FSSP which are reviewed in Hadley and Jones, Structure 7:1099-1112 (1999) and regions having a particular fold or conformation used as a region for sequence comparison to a second polypeptide to identify substantially similar regions. Similarly, functional data including, for example, identification of one or more residues involved with binding or catalysis can be used to locate a region in a sequence alignment for comparison and determination of a substantially similar region.

A polypeptide that is substantially similar to a reference polypeptide can share at least about 70% identity, at least about 80% identity, at least about 90% identity, at least about 95% identity, at least about 97% identity, or at least about 99% identity over the length of the two sequences being compared or in a particular region being compared. As described above, substantially similar sequences can be identified by comparison of one or more particular region such that overall homology between the two sequences is at least about 20% identity over the length of the two sequences being compared. As the ratio of the size of the compared region to the size of the entire polypeptide increases the percent identity will increase to, for example, at least about 30% identity, at least about 40% identity, at least about 50% identity, or at least about 60% identity over the length of the two sequences being compared.

The substitution of functionally equivalent amino acids is routine and can be accomplished by methods known to those skilled in the art. Briefly, the substitution of functionally equivalent amino acids can be made by identifying the amino acids which are desired to be changed, incorporating the changes into the encoding nucleic acid using methods described for example in Sambrook et al., supra (1989); Sambrook et al., supra (2001) and Ausubel et al., supra (1999)) and then determining the function of the recombinantly expressed and modified polypeptide.

An activity of a paraoxonase polypeptide can be determined using a variety of assays. Such enzyme assays can involve detecting the conversion of a paraoxonase substrate to a product by determining an increase in an amount of product generated or a decrease in an amount of substrate consumed. A substrate or product can be detected by characteristic physicochemical properties, such as mass, polarity, charge, light absorption, fluorescence or combinations thereof. For example, paraoxonase activity can be measured in a calorimetric assay in which hydrolysis of phenylacetate by paraoxonase arylesterase activity is determined from increased absorption at 270 nm as described, for example, in Shih et al., J. Clin. Invest. 97:1630-1639 (1996). Other colorimetric methods include measuring hydrolysis of paraoxon to 4-nitrophenol as an increase in absorbance at 412 nm as described, for example, in Watson et al., J. Clin. Invest. 96:2882-2891 (1995). Such assays can be used to determine an activity of a paraoxonase including, for example, binding affinity or catalytic rate constant using well known analyses as described, for example, in Segel, Enzyme Kinetics John Wiley and Sons, New York (1975).

Minor modifications that can occur in a polypeptide while retaining its ability to inhibit a pro-atherogenic molecule activity include, for example, a change made in a region of the polypeptide that does not affect the function. For example, a modification made in a domain of PON1 that does not affect esterase activity can be a minor modification. Various modifications of PON1 and their effects on paraoxonase activity are known in the art as described, for example in Mackness et al., supra (2000). Therefore, a minor modification can include addition of one or more amino acid, addition of one or more moiety, deletion of one or more amino acid, substitution of one or more amino acid or chemical modification of one or more amino acid. Minor modifications can include, for example, attachment of various molecules such as other amino acids, polypeptides, carbohydrates, nucleic acids or lipids.

Minor modifications can also include conservative substitution of one or more amino acids in a polypeptide compared to a reference sequence. Conservative substitutions of encoded amino acids can include, for example, amino acids which belong within the following groups: (1) non-polar amino acids such as Gly, Ala, Val, Leu, and Ile; (2) polar neutral amino acids such as Cys, Met, Ser, Thr, Asn, and Gln; (3) polar acidic amino acids such as Asp and Glu; (4) polar basic amino acids such as Lys, Arg and His; (5) aromatic amino acids such as Phe, Trp, Tyr, and His, and (6) isosteric amino acids such as Ser and Cys. Therefore, a polypeptide of the invention can include sequence variants such as naturally occurring allelic variants or homologs from other organisms so long as the variants retain the ability to inhibit a pro-atherogenic molecule activity.

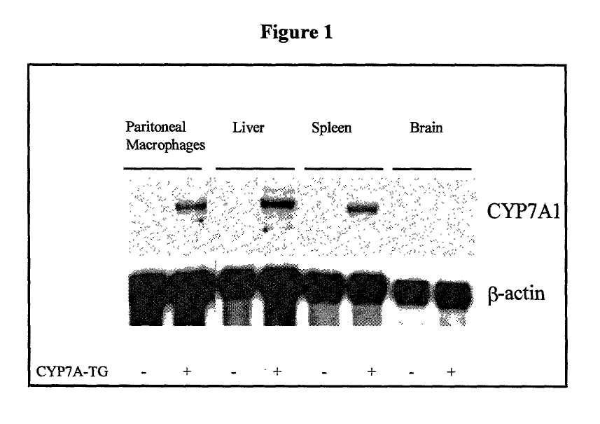

Nucleic acids that have substantially the same sequence can also be identified by the ability to hybridize to each other. Hybridization refers to the binding of complementary strands of nucleic acid, for example, sense:antisense strands or probe:target nucleic acid to each other through Watson-Crick hydrogen bonds. Substantially similar sequences can be identified due to hybridization under conditions of differing stringency including, for example, high stringency, moderate stringency or low stringency. Those skilled in the art can readily determine conditions for hybridization that are appropriate for a particular application including, for example, Northern blot analysis as described in Examples I and II and shown in FIGS. 1, 2 and 5. Conditions of equivalent stringency can be determined by comparison to reference conditions such as those described below.

High stringency hybridization refers to conditions that permit hybridization of only those nucleic acid sequences that form stable hybrids in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5×Denhart's solution, 5×SSPE, 0.2% SDS at 42° C., followed by washing in 0.1×SSPE, and 0.1% SDS at 65° C. Denhart's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA).

Moderate stringency conditions refers to conditions that permit hybridization of only those nucleic acid sequences that form stable hybrids in 50% formamide, 5×Denhart's solution, 5×SSPE, 0.2% SDS at 42° C., followed by washing in 0.2×SSPE and 0.2% SDS, at 42° C. If a hybrid is not stable in these conditions, it will not be stable under moderate stringency conditions, as contemplated herein.

Low stringency hybridization refers to conditions that permit hybridization of only those nucleic acid sequences that form stable hybrids in 10% formamide, 5×Denhart's solution, 6×SSPE, 0.2% SDS at 22° C., followed by washing in 1×SSPE, 0.2% SDS, at 37° C. If a hybrid is not stable in these conditions, it will not be stable under low stringency conditions, as contemplated herein.

Other suitable hybridization conditions that are equivalent to those described above are well known to those of skill in the art and are described, for example, in Sambrook et al., supra (1989); Sambrook et al., supra (2001) and Ausubel et al., supra (1999)).

Nucleic acids having substantially similar sequences can be identified by known methods of sequence comparison including, for example, a BLAST 2.0 alignment using default parameters. Substantially similar sequences can have at least 60%, at least 70%, at least 80%, at least 90%, at least 95%, or at least 98% identity. Substantially similar nucleic acid sequences can also be identified according to substantial similarity of the amino acid sequences and functional activities of the polypeptides they encode.

The invention also provides vectors containing a nucleic acid of the invention including, for example, a nucleic acid encoding a paraoxonase polypeptide, apolipoprotein A1 polypeptide, or cholesterol-7α-hydroxylase polypeptide. Appropriate expression vectors include those that are replicable in eukaryotic cells and/or prokaryotic cells and those that remain episomal or those which integrate into the host cell genome. Suitable vectors for expression in prokaryotic or eukaryotic cells are well known to those skilled in the art (see, for example, Ausubel et al., supra, 1999). The vectors of the invention can be used for subcloning or amplifying a nucleic acid encoding an inhibitor of a pro-atherogenic molecule or for recombinantly expressing a an inhibitor of a pro-atherogenic molecule. A vector of the invention can include, for example, a viral vector such as a bacteriophage, a baculovirus or a retrovirus; cosmid or plasmid; and, particularly for cloning large nucleic acid molecules, bacterial artificial chromosome vectors (BACs) and yeast artificial chromosome vectors (YACs). Such vectors are commercially available, and their uses are well known in the art. One skilled in the art will know or can readily determine an appropriate vector for expression in a particular host cell.

Suitable expression vectors include those capable of expressing a nucleic acid operatively linked to a regulatory sequence or element such as a promoter region or enhancer region that is capable of regulating expression of such nucleic acid. For example, a vector of the invention can include a nucleic acid encoding a an inhibitor of a pro-atherogenic molecule operationally linked to a macrophage-specific expression element. Promoters or enhancers, depending upon the nature of the regulation, can be constitutive, suppressible or inducible. The regulatory sequences or regulatory elements are operatively linked to a nucleic acid of the invention such that the physical and functional relationship between the nucleic acid and the regulatory sequence allows transcription of the nucleic acid.

Any of a variety of inducible promoters or enhancers can also be included in a nucleic acid or vector of the invention to allow control of expression of an inhibitor of a pro-atherogenic molecule by added stimuli or molecules. Such inducible systems, include, for example, tetracycline inducible system (Gossen & Bizard, Proc. Natl. Acad. Sci. USA, 89:5547-5551 (1992); Gossen et al., Science, 268:1766-1769 (1995); Clontech, Palo Alto, Calif.); metalothionein promoter induced by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse mammory tumor virus (MMTV) induced by steroids such as glucocortocoid and estrogen (Lee et al., Nature, 294:228-232 (1981); and heat shock promoters inducible by temperature changes.

An inducible system particularly useful for therapeutic administration utilizes an inducible promotor that can be regulated to deliver a level of therapeutic product in response to a given level of drug administered to an individual and to have little or no expression of the therapeutic product in the absence of the drug. One such system utilizes a Gal4 fusion that is inducible by an antiprogestin such as mifepristone in a modified adenovirus vector (Burien et al., Proc. Natl. Acad. Sci. USA, 96:355-360 (1999). Another such inducible system utilizes the drug rapamycin to induce reconstitution of a transcriptional activator containing rapamycin binding domains of FKBP12 and FRAP in an adeno-associated virus vector (Ye et al., Science, 283:88-91 (1999)). It is understood that any combination of an inducible system can be combined in any suitable vector, including those disclosed herein. Such a regulatable inducible system is advantageous because the level of expression of the therapeutic product can be controlled by the amount of drug administered to the individual or, if desired, expression of the therapeutic product can be terminated by stopping administration of the drug.

Vectors useful for therapeutic administration of a nucleic acid encoding an inhibitor of a pro-atherogenic molecule can contain a regulatory element that provides tissue specific expression of an operatively linked sequence encoding an inhibitor of a pro-atherogenic molecule. In one embodiment, the invention provides a nucleic acid having a sequence encoding an inhibitor of a pro-atherogenic molecule operationally linked to a sequence of a macrophage-specific expression element. A macrophage-specific expression element included in a nucleic acid of the invention can be a macrophage-specific promoter such as a class A scavenger receptor promoter. A nucleic acid of the invention can include a sequence of a macrophage-specific enhancer such as a class A scavenger receptor enhancer. The expression elements can be used individually or in various combinations to suit a particular application of the methods. For example, when used in a therapeutic method, a macrophage specific enhancer can be useful to upregulate expression of an inhibitor of a pro-atherogenic molecule in a macrophage. The absence of enhancer activation or the effect of a silencer in a non macrophage cell can help prevent expression from occurring in non macrophage cells. In this way tissue specific expression elements can provide targeted expression of an inhibitor of a pro-atherogenic molecule.

Expression of an inhibitor of a pro-atherogenic molecule in hepatic cells can occur by use of tissue specific expression elements as well. For example, a nucleic acid encoding an inhibitor of a pro-atherogenic molecule can be operatively linked to an apolipoprotein E promoter element. As described in Simonet, J. Biol. hem., 268:8221-8229 (1993) an apolipoprotein E promoter element allows expression of an operationally attached gene primarily in hepatic cells.

A nucleic acid encoding an inhibitor of a pro-atherogenic molecule can be delivered into a mammalian cell, either in vivo or in vitro using suitable vectors well-known in the art. Suitable vectors for delivering a nucleic acid encoding an inhibitor of a pro-atherogenic molecule to a mammalian cell, include viral vectors such as retroviral vectors, adenovirus, adeno-associated virus, lentivirus, herpesvirus, as well as non-viral vectors such as plasmid vectors. Such vectors are useful for providing therapeutic amounts of an inhibitor of a pro-atherogenic molecule (see, for example, U.S. Pat. No. 5,399,346, issued Mar. 21, 1995).

Viral based systems provide the advantage of being able to introduce relatively high levels of the heterologous nucleic acid into a variety of cells. Suitable viral vectors for introducing an invention nucleic acid encoding an inhibitor of a pro-atherogenic molecule into a mammalian cell are well known in the art. These viral vectors include, for example, Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et_al., Proc. Natl. Acad. Sci. USA, 85:6460-6464 (1988); Blaese et al., Science, 270:475-479 (1995); Onodera et al., J. Virol., 72:1769-1774 (1998)); denovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med., 5:439-443 (1999); Wang et al., Proc. Natl. Acad. Sci. USA, 96:3906-3910 (1999); Snyder et al., Nature Med., 5:64-70 (1999); Herzog et al., Nature Med., 5:56-63 (1999)); retrovirus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997)).

For example, in one embodiment of the present invention, adenovirus-transferrin/polylysine-DNA (TfAdpl-DNA) vector complexes (Wagner et al., Proc. Natl. Acad. Sci., USA, 89:6099-6103 (1992); Curiel et al., Hum. Gene Ther., 3:147-154 (1992); Gao et al., Hum. Gene Ther., 4:14-24 (1993)) can be employed to transduce mammalian cells with a nucleic acid encoding an inhibitor of a pro-atherogenic molecule. Any of the plasmid expression vectors described herein can be employed in a TfAdpl-DNA complex.

A vector of the invention can further contain a selectable marker in order to provide a selectable phenotype for a cell transduced with a nucleic acid encoding an inhibitor of a pro-atherogenic molecule. A selectable marker is generally a gene encoding a product that provides resistance to an agent that inhibits cell growth or kills a cell. A variety of selectable markers can be used in a vector of the invention, including, for example, Neo, Hyg, hisD, Gpt and Ble genes, as described, for example in Ausubel et al. (Current Protocols in Molecular Biology (Supplement 47), John Wiley & Sons, New York (1999)) and U.S. Pat. No. 5,981,830. Drugs useful for selecting for the presence of a selectable marker includes, for example, G418 for Neo, hygromycin for Hyg, histidinol for hisD, xanthine for Gpt, and bleomycin for Ble (see Ausubel et al., supra, (1999); U.S. Pat. No. 5,981,830). A vector of the invention can incorporate a positive selectable marker, a negative selectable marker, or both (see, for example, U.S. Pat. No. 5,981,830).

Therefore, the invention provides a recombinant macrophage expressing a nucleic acid encoding-an inhibitor of a pro-atherogenic molecule. The recombinant cells can be generated by introducing into a host cell a vector containing a nucleic acid molecule encoding an inhibitor of a pro-atherogenic molecule such as a paraoxonase polypeptide, apolipoprotein A1 polypeptide, or cholesterol-7α-hydroxylase polypeptide, as described above. The recombinant cells can be transduced, transfected or otherwise genetically modified to incorporate a nucleic acid of the invention using well known methods.

Numerous different types of cells can be used to construct recombinant cells that express an inhibitor of a pro-atherogenic molecule. Cell types to be selected for generating the recombinant cells of the invention can be those which are capable of polypeptide synthesis and/or secretion. With the exception of highly specialized cell types, the large majority of cells meet these criteria. For example, red blood cells, which are terminally differentiated cells, have lost their nucleus and ability to express genes and are, therefore, unlikely candidates for the cells of the invention. However, with the exclusion of the few cell types that cannot express a nucleic acid and synthesize a polypeptide such as those characterized above, essentially all other cell types can be used for constructing the modified cell or cell populations of the invention. The actual cell type to be used will, therefore, depend on the intended use of the modified cells by those skilled in the art.

The cell type chosen for modification is selected according to the biological characteristics of the cell and according to gene expression criteria well known in the art. For example, objective criteria such as the ease of culture efficiency, the ease of genetic modification and other routine cellular and molecular manipulations can be used to evaluate and select the cell type for modification. Those cell types which can be passaged for several generations without substantial loss in viability are preferable candidates for expression of an inhibitor of a pro-atherogenic molecule in a therapeutic method of the invention. As will be described further below, such cell types include, for example, both primary cells as well as cell lines. Additionally, criteria such as the proliferation characteristics can also be evaluated for selection of the cell type to be modified.

Cell types are additionally selected according the efficiency with which they can be modified to express an inhibitor of a pro-atherogenic molecule. Cell types that can be readily modified and selected for the expression of the introduced genes by any of a variety of methods known in the art are applicable for constructing the cells of the invention. Availability of promoter and regulatory elements can also be included as a criteria for selecting a particular cell type for modification. Such characteristics and criteria are routine and well know to those skilled in the art.

Various combinations of the above exemplary characteristics as well as other characteristics can additionally be used for selecting a cell type to modify. For example, if the objective is to express a particular level of an inhibitor of a pro-atherogenic molecule using a relatively small number of cells, then a cell type which is efficiently modified and can express high levels of the inhibitor of a pro-atherogenic molecule can be selected to achieve the desired result. In contrast, if cell number is not a limiting factor, then it can be desirable to select the cell type because of favorable growth or proliferation characteristics. Additionally, various expression elements can be utilized to augment or modulate the level of expression of an inhibitor of a pro-atherogenic molecule so as to complement advantageous characteristics or overcome any deficiencies of the selected cell types for modifications. Such criteria and characteristics are well known or can be determined by those skilled in the art.

Exemplary host cells that can be used to express an inhibitor of a pro-atherogenic molecule include primary cells or established cell lines, such as COS, CHO, HeLa, NIH3T3, HEK 293 and PC12 cells. Cells can be from a mammal including, for example, a human, non-human mammal, non-human primate, mouse, rat, pig, cow, dog, cat, or horse. A recombinant cell can be derived from a particular tissue or developmental stage including, for example, a hepatic or liver cell, non-liver cell, blood cell, stem cell such as a pluripotent or hematopoietic stem cell, bone marrow progenitor cell, leukocyte, monocyte or macrophage. The recombinant cell is preferably a nucleated cell. Exemplary host cells also include amphibian cells, such as Xenopus embryos and oocytes; insect cells such as Drosophila, nematode cells such as c. elegans, yeast cells such as Saccharomyces cerevisiae, Saccharomyces pombe, or Pichia pastoris, and prokaryotic cells such as Escherichia coli.

The cellular composition of normal adult human blood is about 95% red blood cells, about 5% platelets, and about 0.1% leukocytes. Leukocytes are composed of about 30-40% mononuclear cells (including lymphocytes, monocytes, stem and progenitor cells, and circulating dendritic cells (cirDC)) and about 60-70% granulocytes (including neutrophils, eosinophils and basophils). The characteristics of each of these cell types that facilitate their identification and isolation, including relative size, density, granularity and presence of cell surface markers, are well known in the art (see, for example, Kuby, Immunology 3^{rd} ed., Freeman & Co., New York (1997)). These cells can be transduced with a nucleic acid of the invention and used directly for expression of an inhibitor of a pro-atherogenic molecule, for example, in a therapeutic method of the invention. Alternatively, following transduction, the cells can be treated with an appropriate growth factor or cytokine to cause differentiation of the cell prior to use in a method of the invention. For example, a recombinant monocyte can be treated with M-CSF to cause differentiation of the cell to a macrophage prior to use in a method of the invention.

Thus, a cell used in the methods of the invention can be produced by differentiating a stem cell, macrophage precursor cell or other amenable cell type to form a cell that has a subset of macrophage characteristics including factors that are sufficient for localization to an atherosclerotic plaque and are naturally associated with a macrophage. A cell having a subset of macrophage characteristics can include any macrophage characteristics including, for example, those described above, so long as it can be localized to an atherosclerotic plaque. A cell having a subset of macrophage characteristics can include, for example, the characteristic of providing expression of a nucleic acid under the control of a macrophage-specific expression element. Thus, an inhibitor of a pro-atherogenic molecule can be specifically localized to an atherosclerotic plaque by expressing the inhibitor in a cell having a subset of macrophage characteristics that are sufficient for localization to an atherosclerotic plaque. Alternatively, localization of the gene product can be provided by the atherosclerotic plaque localization factors that are present in the cell and expression can be controlled by a tissue specific or non-tissue specific expression element. Thus, a wide variety of expression elements can be used and the methods do not require tissue specific expression elements. As described above, a recombinant macrophage can also be used to similar advantage with a non-tissue specific expression element since macrophage-specific expression is determined by expression in recombinant macrophages generated in vitro.

Cell types described herein can be obtained by methods known in the art, including density gradient separation through media such as Ficoll or Percoll, apheresis, and positive and negative selection methods (e.g. immunomagnetic selection or flow cytometry), alone or in any combination. Apheresis is a preferred method to remove large numbers of blood cells of a particular type (e.g. peripheral blood mononuclear cells or platelets) from an individual, while returning red blood cells. Cell separators suitable for apheresis and their uses are well known in the art, and include, for example, the FENWAL CS 3000™ cell separator (Baxter International Inc, Deerfield, Ill.), the HAEMONETICS MCS™ system (Haemonetics Corp., Braintree, Mass.), and the COBE Spectra Apheresis System™ (Gambro BCT). A preferred method of further selection of desired cell subsets is immunomagnetic selection using an automated cell selection system, such as an ISOLEX 300i™ cell selection device (Nexell Therapeutics Inc., Irvine Calif.).

The invention further provides a transgenic non-human mammal containing recombinant cells containing a transgenic nucleic acid encoding an inhibitor of a pro-therogenic molecule A recombinant non-human mammal of the invention can be advantageously used in drug screening methods to determine, for example, potential side effects, cross-reactivity and toxicity associated with a drug that increases the activity of an inhibitor of a pro-atherogenic molecule. Drug effects that are unrelated to increased levels of an inhibitor of a pro-atherogenic molecule can be identified by comparing drug-treated control animals with transgenic animals expressing the inhibitor of a pro-atherogenic molecule. For example, physical signs and symptoms of systemic or organ- or tissue-specific toxicity; drug action at undesired target cells, tissues, or organs, and other unpredicted or unexpected physical changes due to drug activities unrelated to increased levels of an inhibitor of a pro-atherogenic molecule can be identified.

A non-human transgenic animal can be treated with a drug before or after occurrence of atherosclerotic lesions or other signs of disease. When a drug is administered before the occurrence of a lesion, the transgenic animal can be used to determine the prophylactic effect of the drug. When a drug is administered to a non-human transgenic animal of the invention after the occurrence of an observable sign or symptom of disease, such an animal can be used, for example, to examine the effect of the a drug on ameliorating atherosclerosis.

An invention non-human transgenic animal can also be advantageously used to determine the role of a an inhibitor of a pro-atherogenic molecule in a particular pathological phenotype or condition of an animal model for atherosclerosis used in drug development. For example, a transgenic animal of the invention can be cross-bred with a disease-model animal to determine if expression of an inhibitor of a pro-atherogenic molecule alters the phenotype of disease. In such a cross-breeding method, a transgenic animal expressing an inhibitor of a pro-atherogenic molecule can be bred with an animal having a variety of phenotypes representative of a atherosclerosis, or any other disease phenotype known or suspected to be altered by increased activity of an inhibitor of a pro-atherogenic molecule.

In a particular embodiment, the invention provides a transgenic non-human mammal that is homozygous for a nucleic acid expressing an inhibitor of a pro-atherogenic molecule. A homozygous animal can be identified as having two copies of the transgene for the inhibitor of a pro-atherogenic molecule. In another embodiment, the invention provides a transgenic non-human mammal that is heterozygous for a nucleic acid expressing an inhibitor of a pro-atherogenic molecule, identifiable as having only one allele of the transgene.

The transgenic non-human mammals of the invention can be produced by creating transgenic animals expressing a nucleic acid encoding an inhibitor of a pro-atherogenic molecule using a variety of techniques. Examples of such techniques include the insertion of normal or mutant versions of a nucleic acid encoding an inhibitor of a pro-atherogenic molecule by microinjection, retroviral infection or other means well known to those skilled in the art, into appropriate fertilized embryos to produce a transgenic animal as described, for example, in Hogan et al., Manipulating the Mouse Embryo: A Laboratory Manual, Cold Spring Harbor Laboratory (1986); Hogan et al., Manipulating the Mouse Embryo: A Laboratory Manual, second ed., Cold Spring Harbor Laboratory (1994), and U.S. Pat. Nos. 5,602,299; 5,175,384; 6,066,778; and 6,037,521. Such techniques include, but are not limited to, pronuclear microinjection as described, for example, in U.S. Pat. No. 4,873,191; retrovirus mediated gene transfer into germ lines as described, for example, in Van der Putten et al., Proc. Natl. Acad. Sci. USA 82:6148-6152 (1985); gene targeting in embryonic stem cells as described, for example, in Thompson et al., Cell 56:313-321 (1989); electroporation of embryos as described, for example, in Lo, Mol Cell. Biol. 3:1803-1814 (1983); and sperm-mediated gene transfer as described, for example, in Lavitrano et al., Cell 57:717-723 (1989).

Different methods can be used to introduce a transgene depending on the stage of development of the embryonal cell. The zygote is a good target for micro-injection, and methods of microinjecting zygotes are well known (see U.S. Pat. No. 4,873,191). In the mouse, the male pronucleus reaches the size of approximately 20 micrometers in diameter which allows reproducible injection of 1-2 picoliters (pl) of DNA solution. The use of zygotes as a target for gene transfer has a major advantage in that in most cases the injected DNA will be incorporated into the host genome before the first cleavage (see Brinster, et al. Proc. Natl. Acad. Sci. USA 82:4438-4442 (1985)). As a consequence, all cells of the transgenic non-human animal will carry the incorporated transgene. This will, in general, also be reflected in the efficient transmission of the transgene to offspring of the founder since 50% of the germ cells will harbor the transgene.

The transgenic animals of the present invention can also be generated by introduction of the targeting vectors into embryonal stem (ES) cells. ES cells are obtained by culturing pre-implantation embryos in vitro under appropriate conditions as described, for example, in Evans et al., Nature 292:154-156 (1981); Bradley et al., Nature 309:255-258 (1984); Gossler et al., Proc. Natl. Acad. Sci. USA 83:9065-9069 (1986); and Robertson et al., Nature 322:445-448 (1986). Transgenes can be efficiently introduced into ES cells by DNA transfection using a variety of methods known in the art including electroporation, calcium phosphate co-precipitation, protoplast or spheroplast fusion, lipofection and DEAE-dextran-mediated transfection. Transgenes can also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (reviewed in Jaenisch, Science 240:1468-1474 (1988)). Prior to the introduction of transfected ES cells into the blastocoel, the transfected ES cells can be subjected to various selection protocols to enrich for those that have integrated the transgene if the transgene provides a means for such selection. Alternatively, PCR can be used to screen for ES cells that have integrated the transgene. This technique obviates the need for growth of the transfected ES cells under appropriate selective conditions prior to transfer into the blastocoel.

Retroviral infection can also be used to introduce a transgene into a non-human animal. The developing non-human embryo can be cultured in vitro to the blastocyst stage. During this time, the blastomeres can be targets for retroviral infection, for example, using methods described in Janenich, Proc. Natl. Acad. Sci. USA 73:1260-1264 (1976). Efficient infection of the blastomeres is obtained by enzymatic treatment to remove the zona pellucida as described, for example, in Hogan et al., supra, 1986. The viral vector system used to introduce the transgene is typically a replication-defective retrovirus carrying the transgene as described, for example, in Jahner et al., Proc. Natl. Acad Sci. USA 82:6927-6931 (1985), and Van der Putten, et al. Proc. Natl. Acad Sci. USA 82:6148-6152 (1985). Transfection is easily and efficiently obtained by culturing the blastomeres on a monolayer of virus-producing cells as described, for example, in Van der Putten, supra, 1985, and Stewart et al., EMBO J. 6:383-388 (1987). Alternatively, infection can be performed at a later stage. Virus or virus-producing cells can be injected into the blastocoele as described, for example, in Jahner D. et al., Nature 298:623-628 (1982). Most of the founders will be mosaic for the transgene since incorporation occurs only in a subset of cells which form the transgenic animal. Further, the founder can contain various retroviral insertions of the transgene at different positions in the genome, which generally will segregate in the offspring. In addition, it is also possible to introduce transgenes into the germline by intrauterine retroviral infection of the idgestation embryo as described, for example, in Jahner t al., supra, 1982. Additional means of using retroviruses or retroviral vectors to create transgenic animals known to the art involves the micro-injection of retroviral particles or mitomycin C-treated cells producing retrovirus into the perivitelline space of fertilized eggs or early embryos as described, for example, in WO 90/08832 (1990), and Haskell and Bowen, Mol. Reprod. Dev. 40:386 (1995).

A nucleic acid encoding an inhibitor of a pro-atherogenic molecule can be microinjected into single-cell embryos in non-human mammals such as a mouse as described in Example I. Using this method, the injected embryos are transplanted to the oviducts/uteri of pseudopregnant females and finally transgenic animals are obtained.

Once the founder animals are produced, they can be bred, inbred, outbred, or crossbred to produce colonies of the particular animal. Examples of such breeding strategies include but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic mice to produce mice homozygous for a given integration site in order to both augment expression and eliminate the need for screening of animals by DNA analysis; crossing of separate homozygous lines to produce compound heterozygous or homozygous lines; breeding animals to different inbred genetic backgrounds so as to examine effects of modifying alleles on expression of the transgene and the effects of expression on susceptibility to developing a hypercholesterolemia-associated condition.

The present invention provides transgenic non-human mammals that carry the transgene encoding an inhibitor of a pro-atherogenic molecule in all their cells, as well as animals that carry the transgene in some, but not all their cells, that is, mosaic animals.

The transgene can be integrated as a single transgene or in concatamers, for example, head-to-head tandems or head-to-tail tandems. In addition, the transgene can be integrated at multiple sites. The integration of multiple transgenes can provide increased expression levels for an inhibitor of a pro-atherogenic molecule. Thus, the methods provide transgenic non-human animals, and cells derived therefrom having different expression levels. Different transgenic non-human animals, or cells derived therefrom, can be assayed using methods described above to identify those having a desired expression level for a particular therapeutic or diagnostic application.

A transgenic animal of the invention can be any non-human mammal such as a mouse, including particular strains described herein, a rabbit, goat, pig, guinea pig, sheep, cow, non-human primate or any non-human mammal. It is understood that animals expressing a transgene for an inhibitor of a pro-atherogenic molecule, in addition to the C57BL/6J strain disclosed herein, can be used as an animal model for reduced susceptibility to hypercholesterolemia-associated disease.

A transgenic non-human mammal of the invention can be a C57BL/6J strain mouse. The C57BL/6J strain develops atherosclerotic lesions and cholesterol gallstones when fed an atherogenic diet containing high cholesterol (Dueland et al., J. Lipid Res., 34:923-931 (1993); Paigen et al., Proc. Natl. Acad. Sci. USA, 84:3763-3767 (1987); Paigen et al., Genetics, 122:163-168 (1989); Dueland et al., J. Lipid Res., 38:1445-1453 (1997); Machleder et al., J. Clin. Invest., 99:1406-1419 (1997); Khanuja et al., Proc. Natl. Acad. Sci. USA, 92:7729-7733 (1995); Wang et al., J. Lipid. Res., 38:1395-1411 (1997); Wang et al., J. Lipid. Res., 40:2066-2079 (1999); Miyake et al., J. Biol. Chem., 275:21805-21808 (2000)). In response to this atherogenic diet, C57BL/6J mice display reduced expression of CYP7A1, an accumulation of atherogenic plasma lipoproteins including very low density lipoprotein (VLDL), intermediate density lipoprotein (IDL) and low density lipoprotein (LDL) (Paigen et al., Atherosclerosis, 57:65-73 (1985)) and reduced plasma high density lipoprotein (HDL) levels (Paigen et al., Proc. Natl. Acad. Sci. USA, 84:3763-3767 (1987); Machleder et al., J. Clin. Invest., 99:1406-1419 (1997)) and inflammatory responses that occur both within the liver (Liao et al., J. Clin. Invest., 91:2572-2579 (1993); Liao et al., J. Clin. Invest., 94:877-884 (1994)) and the arterial wall (Shi et al., Circ. Res., 86:1078-1084 (2000)).

An atherogenic diet is a food preparation that contains higher amounts of cholesterol or other pro-atherosclerotic lipids than a standard or normal diet. As described herein, in Example II, an atherogenic diet suitable for mice contains 1.25% cholesterol. An atherogenic diet can consists of normal Purina breeder chow supplemented with cholesterol. Synthetic low and high fat diets for the study of atherosclerosis in the mouse are described, for example, in Nishina, et al. (J. Lipid Res. 31:859-869 (1990)). Atherogenic diets suitable for a variety of mammalian species, including, for example, the mouse, hamster, rabbit, swine and monkey, are known to those skilled in the art. Such diets can be readily prepared using easily obtained ingredients and can be obtained commercially (for example, from ICN Biomedicals, Aurora, Ohio; Dyets, Inc., Bethlehem, Pa.; and Harlan-Teklad, Indianapolis, Ind.).

An atherogenic diet can be fed to an animal to induce various degrees of atherosclerosis or atherosclerosis-associated symptom or characteristic. For example, feeding C57BL/6J mice such a diet for a longer time period, such as greater than about 20 weeks, will generally produce more severe atherosclerosis than feeding for shorter time periods, such as fewer than 8 weeks. Those skilled in the art can determine the appropriate length of time for administering a particular diet or other atherogenic treatment in order to produce a particular disease characteristic. Those skilled in the art will recognize that disease development and progression will differ among various animal strains and species and will know how to select an appropriate physiological or biochemical endpoint, including, for example, those described herein, for assessing atherosclerosis in a transgenic animal.

A variety of mouse strains well known in the art exhibit similar susceptibility to developing atherosclerotic lesions when fed an atherogenic diet. For example, apoE-deficient mice, LDL receptor-deficient mice and several inbred strains develop atherosclerotic lesions when fed an atherogenic diet (Ragendra et al. J. Lipid Research, 36:2320-2328 (1995) and Paigen B. Am. J. Clin. Nutr., 62:458S-462S (1995)). A transgene encoding an inhibitor of a pro-atherogenic molecule can be similarity introduced into such mice to produce animal models of reduced susceptibility to atherosclerosis.

A transgenic animal or recombinant cell expressing an inhibitor of a pro-atherogenic molecule, can be screened and evaluated to select those animals or cells having a nucleic acid encoding the inhibitor of a pro-atherogenic molecule. Well known methods can be used to identify the presence or location of the nucleic acid including, for example, Southern blot analysis or PCR techniques on genomic DNA isolated from a cell or tissue.

A transgenic animal or recombinant cell of the invention can also be identified or selected according to the level at which a nucleic acid encoding an inhibitor of a pro-atherogenic molecule is expressed. Expression level can be determined by quantitating expression of an mRNA product of a transgene in a cell or tissue using techniques which include, but are not limited to, Northern blot analysis, in situ hybridization analysis, nuclease protection and reverse transcriptase-PCR (RT-PCR). Additionally, expression level can be determined by quantitating the amount of an inhibitor of a pro-atherogenic molecule present in a cell or tissue including, for example, immunochemical methods, such as western blotting, ELISA or immunoprecipitation using an antibody specific for the inhibitor of a pro-atherogenic molecule; detection of fused reporter polypeptide such as a polyhistidine tag (Qiagen; Chatsworth, Calif.), antibody epitope such as the flag peptide (Sigma; St Louis, Mo.), glutathione-S-transferase (Amersham Pharmacia; Piscataway, N.J.), cellulose binding domain (Novagen; Madison, Wisc.), calmodulin (Stratagene; San Diego, Calif.), staphylococcus protein A (Pharmacia; Uppsala, Sweden), maltose binding protein (New England BioLabs; Beverley, Mass.) or strep-tag (Genosys; Woodlands, Tex.). An antibody for detecting an inhibitor of a pro-atherogenic molecule can be made and used according to well known methods as described, for example in, Harlow and Lane, Antibodies: A Laboratory Manual, Cold Spring Harbor Laboratory, New York (1989). A reporter polypeptide can be fused to an inhibitor of a pro-atherogenic molecule using well known cloning methods including those described by the respective manufacturers indicated above.

Selection of a transgenic non-human animal or recombinant cell having a nucleic acid encoding an inhibitor of a pro-atherogenic molecule can also be based on activity of the inhibitor of a pro-atherogenic molecule. Activity can be measured using an assay for the inhibitor of a pro-atherogenic molecule, such as those described above, on a tissue, fluid, cell or subcellular fraction. Although a variety of assays are suitable for measuring activity in crude fractions, an inhibitor of a pro-atherogenic molecule can be isolated from other biological components or purified to homogeneity prior to assaying activity. An inhibitor of a pro-atherogenic molecule can be isolated by well known methods of fractionation including, for example, those described in Scopes, Protein Purification: Principles and Practice, 3^{rd} Ed., Springer-Verlag, New York (1994); Duetscher, Methods in Enzymology, Vol 182, Academic Press, San Diego (1990), and Coligan et al., Current protocols in Protein Science, John Wiley and Sons, Baltimore, Md. (2000). The course of purification and identification of fractions containing an inhibitor of a pro-atherogenic molecule can be determined by immunological detection or activity assay.

A transgenic non-human mammal expressing an inhibitor of a pro-atherogenic molecule can be identified or characterized according to an anti-atherosclerotic phenotype. An anti-atherosclerotic phenotype can be characterized by reduced number or size of atherosclerotic lesions in an animal fed an atherogenic diet using methods such as those described in Example II. Other methods for characterizing and quantitating atherosclerosis in mammals are well known in the art and are described, for example, in Tangirala, et al. (J. Lipid Research, 36:2320-2328 (1995)) and Paigen et al. (Atherosclerosis, 68:231-240 (1987)).

The invention further provides a method for inhibiting or reducing atherosclerosis including administering to an individual a population of recombinant cells expressing a nucleic acid encoding an inhibitor of a pro-atherogenic molecule. The methods can be used to treat any individual at risk for developing atherosclerosis or presenting symptoms associated with atherosclerosis. Those skilled in the art will know or be able to determine risk factors for developing atherosclerosis including, for example, the presence of one or more gene or allele genetically associated with the condition, diet, or level of physical activity. Symptoms of atherosclerosis include, for example, those described previously herein. The particular combination of symptoms and/or risk factors that identify an individual to be treated by the methods can differ. For example, although high blood cholesterol can identify an individual at risk for developing atherosclerosis, an individual having a particular atherosclerosis associated allele can be at risk for developing the condition even when cholesterol levels are within a range considered normal for the general population. The appropriate symptoms and/or risk factors for identifying a particular individual to be treated by the methods of the invention can be readily determined by those skilled in the art.

An isolated cell can be transfected with a nucleic acid encoding a paraoxoanse polypepdite using methods described above. The cells can be tested using routine assays for expression level, secretion or activity to identify cells that are appropriate for administration to a particular individual. Thus, cells having differing expression levels, for example, due to differences in location of genomic insertion (also known in the art as positional cloning effects), can be screened and a cell or population of cells having an optimum or desired expression level selected. Similar screening can be used to test different expression elements or different orientations of particular expression elements such that those producing a gene product at a desired level can be selected. Additionally, when an inducible promoter is used, the cells can be tested in vitro for response to a particular inducing agent to identify an appropriate dose of the inducing agent for administration to an individual prior to administering the cells. Based on expression levels observed in vitro, the number of cells to be administered can also be determined. Therefore, a therapeutic approach using ex vivo gene transfer can provide the advantage of prescreening the cells thereby insuring targeted delivery of the gene to the desired cell, and determining appropriate levels of the expressed gene product.

For therapeutic applications, a cell population can be chosen to be administered to an individual and remain viable in vivo without being substantially rejected by the host immune system. Those skilled in the art know what characteristics should be exhibited by cells to remain viable following administration. Moreover, methods well known in the art are available to augment the viability of cells following administration to a recipient individual.

One characteristic that can be exhibited by the cell or cell population to be administered is that they are substantially immunologically compatible with the recipient individual. A cell is immunologically compatible if it is either histocompatible with recipient host antigens or if it exhibits sufficient similarity in cell surface antigens so as not to elicit an effective host anti-graft immune response. Specific examples of immunologically compatible cells include autologous cells isolated from an individual to be treated and allogeneic cells which have substantially matched major histocompatibility (MHC) or transplantation antigens with the recipient individual. Immunological compatibility can be determined by antigen typing using methods well known in the art. Using such antigen typing methods, those skilled in the art will know or can determine what level of antigen similarity is necessary for a cell or cell population to be immunologically compatible with a recipient individual. The tolerable differences between a donor cell and a recipient can vary with different tissues and can be readily determined by those skilled in the art.

In addition to selecting cells which exhibit characteristics that maintain viability following administration to a recipient individual, methods well known in the art can be used to reduce the severity of an anti-graft immune response. Such methods can therefore be used to further increase the in vivo viability of immunologically compatible cells or to allow the in vivo viability of less than perfectly matched cells or of non-immunologically compatible cells. Therefore, for therapeutic applications, it is not-necessary to select a cell type from the individual to be treated in order to achieve viability of the modified cell following administration. Instead, and as described further below, alternative methods can be employed which can be used in conjunction with essentially any donor cell to confer sufficient viability of the modified cells to achieve a particular therapeutic effect.

For example, in the case of partially matched or non-matched cells, immunosuppressive agents can be used to render the host immune system tolerable to administration or engraftment of the cells. The regimen and type of immunosuppressive agent to be administered will depend on the degree of MHC similarity between the modified donor cell and the recipient. Those skilled in the art know, or can determine, what level of histocompatibility between donor and recipient antigens is applicable for use with one or more immunosuppressive agents. Following standard clinical protocols, administration and dosing of such immunosuppressive agents can be adjusted to improve efficiency of engraftment and the viability of the cells of the invention. Specific examples of immunosuppressive agents useful for reducing a host anti-graft immune response include, for example, cyclosporin, corticosteroids, and the immunosuppressive antibody known in the art as OKT3.

Another method which can be used to confer sufficient viability on partially-matched or non-matched cells is through the masking of the cells or of one or more MHC antigen(s) to protect the cells from host immune surveillance. Such methods allow the use of non-autologous cells in an individual. Methods for masking cells or MHC molecules are well known in the art and include, for example, physically protecting or concealing the cells, as well as disguising them, from host immune surveillance. Physically protecting the cells can be achieved, for example, by encapsulating the cells within a semi-permeable barrier that allows exchange of nutrients and macro molecules. Such a barrier prevents contact of host immune cells such as T-cells with the cells contained within the semi-permeable barrier but still allows induction and/or secretion of an inhibitor of a pro-atherogenic molecule. Encapsulated cells can therefore be used as an implantable device for providing viable cells producing an inhibitor of a pro-atherogenic molecule. The encapsulated cells can be permanently implanted or periodically replaced depending on the cell type used and the location where the device is implanted. An example of a semi-permeable barrier includes natural or synthetic membranes with a pore size that excludes cell-cell contact. Generally, a pore size of about 0.22 mm is sufficient to allow exchange of macromolecules such as an inhibitor of a pro-atherogenic molecule, inducing agents and growth factors without allowing immune cells access to implanted cells. However, other pore sizes can also be used without affecting viability of the recombinant cells. Alternatively, antigens can be disguised by treating them with binding molecules such as antibodies that mask surface antigens and prevent recognition by the immune system.

Immunologically naive cells can also be used for constructing an inhibitor of a pro-atherogenic molecule producing cells. Immunologically naive cells are devoid of MHC antigens that are recognized by a host anti-graft immune response. Alternatively, such cells can contain one or more antigens in a non-recognizable form or can contain modified antigens that faithfully mirror a broad spectrum of MHC antigens and are therefore recognized as self-antigens by most MHC molecules. The use of immunologically naive cells therefore has the added advantages of circumventing the use of the above-described immunosuppressive methods for augmenting or conferring immunocompatibility onto partially or non-matched cells. As with autologous or allogeneic cells, such immunosuppressive methods can nevertheless be used in conjunction with immunologically naive cells to facilitate viability of the recombinant cells.

An immunologically naive cell, or broad spectrum donor cell, can be obtained from a variety of undifferentiated tissue sources, as well as from immunologically privileged tissues. Undifferentiated tissue sources include, for example, cells obtained from embryonic and fetal tissues. An additional source of immunologically naive cells include stem cells and lineage-specific progenitor cells. These cells are capable of further differentiation to give rise to multiple different cell types. Stem cells can be obtained from embryonic, fetal and adult tissues using methods well known to those skilled in the art. Such cells can be used directly or modified further to enhance their donor spectrum of activity.

Immunologically privileged tissue sources include those tissues which express, for example, alternative MHC antigens or immunosuppressive molecules. A specific example of alternative MHC antigens are those expressed by placental cells which prevent maternal anti-fetal immune responses. Additionally, placental cells are also known to express local immuno-suppressive molecules which inhibit the activity of maternal, immune cells.

An immunologically naive cell or other donor cell can be modified to express genes encoding, for example, alternative MHC or immuno-suppressive molecules which confer immune evasive characteristics. Such a broad spectrum donor cell, or similarly, any of the donor cells described previously, can be tested for immunological compatibility by determining its immunogenicity in the presence of recipient immune cells. Methods for determining immunogenicity and criteria for compatibility are well known in the art and include, for example, a mixed lymphocyte reaction, a chromium release assay or a natural killer cell assay. Immunogenicity can be assessed by culturing donor cells together with lympohocyte effector cells obtained from an individual to be treated and measuring the survival of the donor cell targets. The extent of survival of the donor cells is indicative of, and correlates with, the viability of the cells following administration.

Cells can be administered to an individual by a variety of methods known in the art including, for example, injection into the blood stream or surgical implantation. Direct injection of cells into the blood stream is described in Example II. Administration can occur at various locations in an individual to achieve delivery of an inhibitor of a pro-atherogenic molecule to tissues affected by atherosclerosis. For example, recombinant cells of the invention can be injected proximal to a site particularly susceptible to atherosclerosis, identified as containing a growing plaque, or particularly critical as requiring unoccluded blood flow. Alternatively, a recombinant cell expressing an inhibitor of a pro-atherogenic molecule can be implanted into a location that provides the cell access to the blood stream and in particular an artery affected by atherosclerosis. Recombinant cells can be implanted in the methods of the invention by grafting or administration with other components such as matrix components, fragments or other molecules which facilitate adhesion of the cells. The location for implantation can be chosen according to various other criteria including, for example, the presence of nutrients required for cell viability and the presence of growth factors or cytokines for differentiation of the cell. Accordingly, a monocyte or other macrophage progenitor cell can be implanted into the bone marrow of an individual such that maturation and release of the cells to the blood stream can occur by natural processes.

The invention further provides a method for inhibiting or reducing atherosclerosis including administering to an individual a nucleic acid encoding an inhibitor of a pro-atherogenic molecule, the inhibitor of a pro-atherogenic molecule operationally linked to a macrophage-specific expression element. A cell in an individual can be transduced with a nucleic acid of the invention by methods described above. The use of a macrophage-specific expression element provides targeted expression such that the an inhibitor of a pro-atherogenic molecule is not expressed in non macrophage cells. Targeting of expression can be further augmented by delivery of a nucleic acid of the invention to a particular tissue or fluid. For example, the nucleic acid can be injected directly into a particular tissue or location. Direct injection into the bone marrow can be advantageous for targeted delivery to monocytes or other macrophage progenitor cells. Alternatively, a nucleic acid of the invention can be injected into the blood stream for contact with blood borne macrophages and macrophage progenitor cells.

The invention further provides a method of identifying a compound that reduces susceptibility to developing atherosclerosis. The method includes the steps of (a) contacting a cell expressing an inhibitor of a pro-atherogenic molecule with a candidate compound and a pro-atherogenic molecule, under conditions that allow the inhibitor of a pro-atherogenic molecule to inhibit the pro-atherogenic molecule in the absence of the candidate compound; (b) determining an activity of the pro-atherogenic molecule in the presence of the inhibitor of a pro-atherogenic molecule and the candidate compound; and (c) identifying a compound that decreases activity of the pro-atherogenic molecule in the presence of the inhibitor of a pro-atherogenic molecule, the compound being characterized as a compound that reduces susceptibility to developing atherosclerosis.

A method of identifying a compound that reduces susceptibility to developing atherosclerosis can include the steps of (a) contacting a candidate compound with a cell expressing an inhibitor of a pro-atherogenic molecule; (b) determining an activity of the inhibitor of a pro-atherogenic molecule; and (c) identifying a compound that increases activity of the inhibitor of a pro-atherogenic molecule, the compound being characterized as a compound that reduces susceptibility to developing atherosclerosis.

A cell contacted by a candidate compound in a method of the invention can be an isolated cell or a cell in an in vivo environment, for example, in a transgenic animal. The methods of the invention can include contacting a cell expressing an inhibitor of a pro-atherogenic molecule with a candidate compound and determining a change in expression or activity. Changes in expression or activity of an inhibitor of a pro-atherogenic molecule can be determined using the methods described above. Because increased activity of the inhibitor of a pro-atherogenic molecule is associated with reduced susceptibility to atherosclerosis, a candidate compound that causes an increase in an mRNA encoding an inhibitor of a pro-atherogenic molecule or polypeptide levels or increase in an activity such as esterase activity can be identified as a compound that reduces susceptibility to developing atherosclerosis. Accordingly, a compound identified by the methods of the invention as reducing susceptibility to atherosclerosis can have the effect of increasing transcription of a an inhibitor of a pro-atherogenic molecule mRNA, increasing stability of the mRNA, increasing stability of an inhibitor of a pro-atherogenic molecule, increasing translation of an inhibitor of a pro-atherogenic molecule, altering the structure of an inhibitor of a pro-atherogenic molecule to increase substrate binding or catalysis rate. Molecules that mediate the regulation of expression of an inhibitor of a pro-atherogenic molecule or activity can also be targets of compounds that reduce susceptibility to atherosclerosis. For example, a signal transduction pathway that stimulates the activity of an inhibitor of a pro-atherogenic molecule can be modulated or a protein that inhibits or activates an inhibitor of a pro-atherogenic molecule by post translational modification can be modulated by a compound identified by the methods of the invention.

A compound can directly increase activity of an inhibitor of a pro-atherogenic molecule, for example, by binding to the inhibitor of a pro-atherogenic molecule and increasing catalytic activity, such as by inducing a conformational change or by an allosteric effect. A compound that directly increases the activity of a paraoxoanse polypeptide can be identified by contacting the compound with an isolated or purified paraoxoanse polypeptide. Therefore, the invention provides a method for identifying a compound that reduces susceptibility to developing atherosclerosis including contacting a candidate compound with a an inhibitor of a pro-atherogenic molecule and identifying a compound that increases its activity as a compound that reduces susceptibility to developing atherosclerosis.

An assay method for identifying compounds that increase activity of an inhibitor of a pro-atherogenic molecule can be carried out in comparison to a control. One type of a control useful in a method of the invention is a transgenic animal or recombinant cell expressing an inhibitor of a pro-atherogenic molecule or an isolated inhibitor of a pro-atherogenic molecule that is treated substantially the same as the test animal, cell, or polypeptide exposed to a candidate compound, except that the control is not exposed to a compound. Such a control can be useful to correct for effects that are not due to effects of the compound on an inhibitor of a pro-atherogenic molecule. Another type of control useful in a method of the invention is a cell or animal which does not express an inhibitor of a pro-atherogenic molecule. Such a cell or animal can be used to correct for effects that are not due to the presence of an inhibitor of a pro-atherogenic molecule.

Compounds useful as potential therapeutic agents can be generated by methods well known to those skilled in the art, for example, well known methods for producing pluralities of compounds, including chemical or biological molecules such as simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources. Combinatorial libraries of molecules can be prepared using well known combinatorial chemistry methods (Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997)).

Such libraries can be screened to identify a compound that reduces susceptibility to hypercholesterolemia-associated conditions using assay methods described above. The effectiveness of compounds identified by an initial in vitro screen can be further tested in vivo using animal models of atherosclerosis-associated conditions well known in the art, such as the atherosclerosis mouse models described herein. However, if desired, compounds can be screened using an in vivo assay, for example, using transgenic or non-transgenic animals.

The following examples are intended to illustrate but not limit the present invention.

### EXAMPLE I

### Production of Transgenic Mice Expressing a CYP7A1 or PON1 Polypeptide

This example describes generation of transgenic mouse lines expressing CYP7A1 or PON1 in monocyte/macrophage populations.

The Acetyl-LDL receptor transgenic plasmid was constructed to include sequences for the acetyl LDL receptor (scavenger receptor) expression elements as follows. The vector was constructed to include an insert containing a roughly 4 kb human scavenger receptor enhancer and an 800 bp promoter at the 5′ end. The 3′ end contains 1 kb encompassing exons 3, 4, 5 of the human growth hormone including the poly (A) tail. The vector containing the insert was pBluescriptIIKS (Stratagene; La Jolla, Calif.).

The rat CYP7A1 cDNA (1.8 kb) was excised from pcDNA3-7alpha with EcoRI and ligated into the transgenic polylinker of Acetyl-LDL receptor transgenic plasmid at the EcoRI site of the pBluescriptIIKS located downstream of the scavenger receptor expression elements. Restriction mapping and sequencing were used to confirm orientation of the insert. The vector was then excised from the plasmid with XhoI at the 5′ end and NotI at the 3′ end. The QIAquick gel extraction kit (Qiagen) was used to isolate the transgenic vector from bacterial sequences.

The PON1 transgenic vector was generated as follows. The mouse PON-1 cDNA (1.5kb) was excised from mousePONcDNA#5 with EcoRI and PvuII. The Acetyl-LDL receptor transgenic plasmid was digested with EcoRI and EcoRV. The PON-1 cDNA was then ligated into the plasmid. The vector was then excised from the plasmid with XhoI at the 5′ end and NotI at the 3′ end. Restriction mapping and sequencing were used to confirm orientation of the insert. The QIAquick gel extraction kit (Qiagen) was used to isolate the transgenic vector from bacterial sequences.

The constructs were separately microinjected into single cell embryos of C57BL/6 mice and implanted into pseudo-pregnant female mice. As shown in FIG. 1, a founder group was found to express CYP7A1 mRNA using a ribonuclease protection assay that distinguished the rat transgene CYP7A1 from the endogenous mouse CYP7A1. The expression of the CYP7A1 was exclusively in tissues containing a significant population of macrophages (spleen, liver and peritoneal macrophages obtained from thioglycolate-induced mice), but not in brain as shown in FIG. 1.

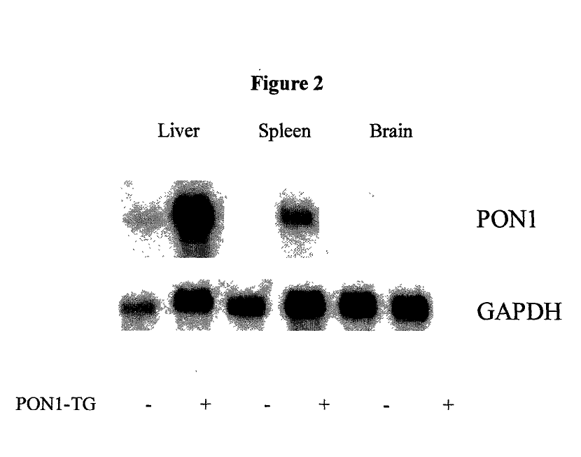

Founder C57BL/6 mice bearing the PON1 transgene were bred and their progeny screened for the expression of PON1 mRNA. In non-transgenic mice the expression of the endogenous PON1 mRNA was present in liver, but not detected in spleen or brain. In PON1 transgenic mice the expression of PON1 mRNA was markedly increased in the liver and spleen, but not in the brain as shown in FIG. 2. These data demonstrate that the PON1 transgene was expressed by macrophages.

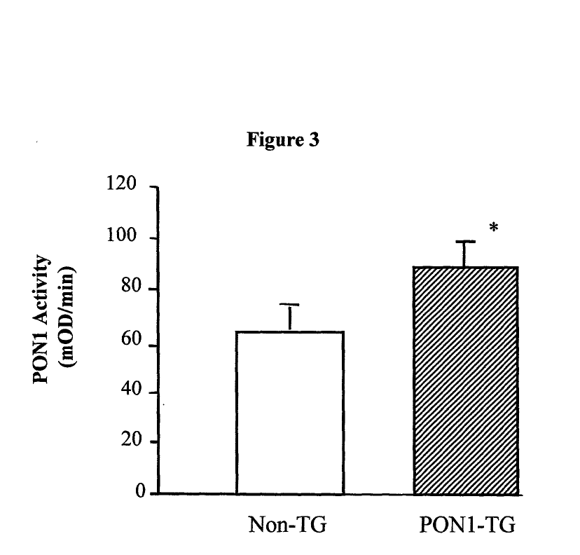

As shown in FIG. 3, the enzymatic activity of PON1 in the plasma of transgenic mice used for bone marrow transplantation was 30% greater (p<0.01) than the activity in plasma of non-transgenic littermates. This data demonstrates that the transgene increased PON1 enzymatic activity in the circulation.

Thus, transgenic C57BL/6J mice that express PON1 or CYP7A1 in a tissue specific manner in monocyte/macrophages were generated.

### EXAMPLE II

### Reduction of Atherosclerosis in Transgenic Mice Expressing a PON1 or CYP7A1 Polypeptide

This Example demonstrates administration of PON1 or CYP7A1 expressing cells to mice and significant reduction of atherosclerotic lesion formation in the mice due to presence of the cells.

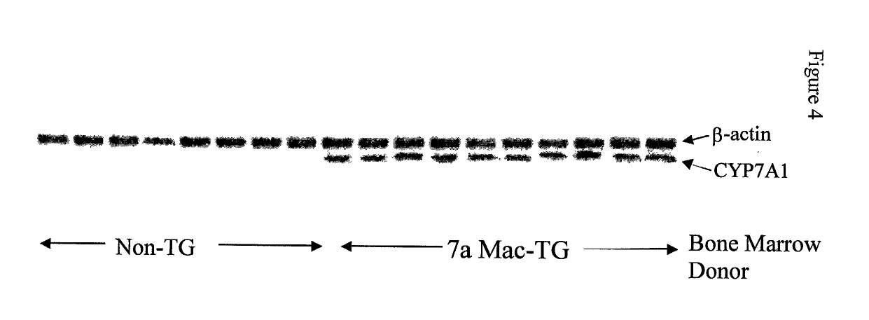

Bone marrow obtained from mice expressing the CYP7A1 transgene was injected into lethally irradiated C57BL/6 LDL receptor−/−mice. Control mice received bone marrow from non-transgenic littermates. One month later, circulating white blood cells were obtained and analyzed for the presence of the CYP7A1 mRNA using RT-PCR. All mice that received bone marrow from CYP7A1 mice showed the presence of CYP7A1 mRNA in their white blood cells, whereas no CYP7A1 mRNA was detected in cells obtained from mice receiving bone marrow from non-transgenic littermates as shown in FIG. 4. These data demonstrate that stem cells bearing the CYP7A1 transgene were delivered to the recipient mice in a manner that allowed its expression in circulating white blood cells.

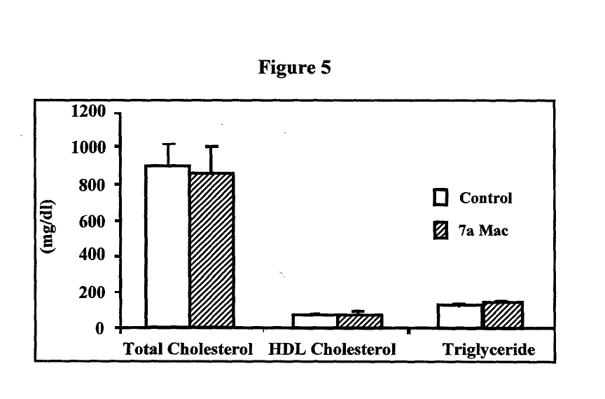

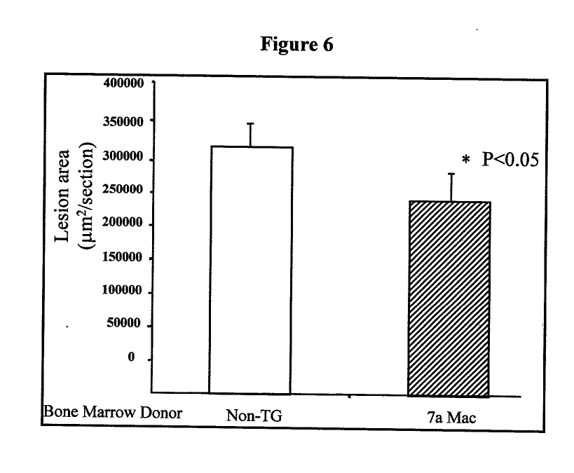

The mice were placed on an atherogenic diet containing 1.25% cholesterol (TD96335; Harlan Teklad) for 20 weeks. After this time mice were sacrificed and their plasma lipids and atherosclerosis were quantitated. While the plasma levels of triglycerides, total cholesterol and HDL cholesterol were similar in both groups of mice (FIG. 5), mice receiving bone marrow from CYP7A1 mice showed a ˜22% statistically significant (p<0.05) reduction in atherosclerosis lesions (FIG. 6).

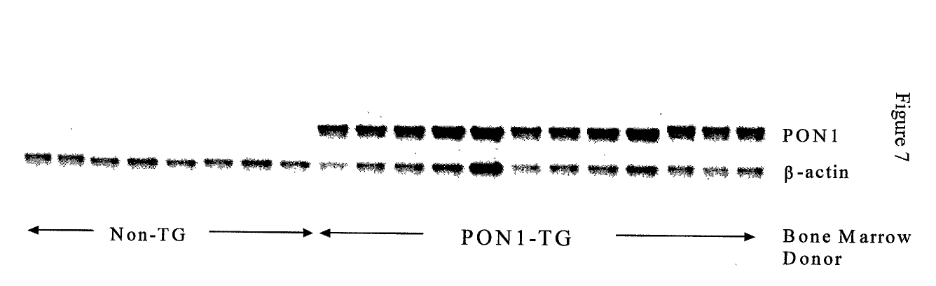

Bone marrow from PON1 transgenic and non-transgenic littermates was transplanted into irradiated LDL receptor−/− mice. RT-PCR of mRNA extracted from white blood cells obtained one month after bone marrow transplantation showed the presence of PON1 mRNA in mice receiving bone marrow from the PON1 transgenic mice, whereas no PON1 mRNA was detected in control mice receiving bone marrow from non-transgenic littermates (FIG. 7).

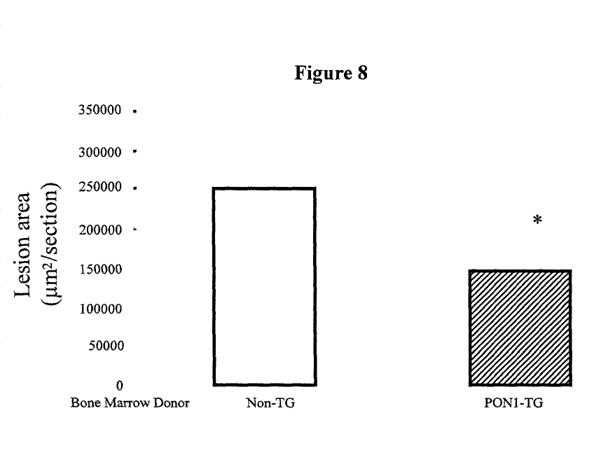

The PON1 and littermate control mice were placed on an atherogenic diet containing 1.25% cholesterol (TD96335; Harlan Teklad) for 16 weeks. Plasma levels of total cholesterol, HDL cholesterol and triglycerides were similar for both groups of mice throughout the entire experiment. Mice were sacrificed and atherosclerosis lesions were quantified using oil red O staining. Mice receiving bone marrow from PON1 transgenic mice displayed a significant 40% reduction in atherosclerosis lesions, P<0.001 as shown in FIG. 8. These data demonstrate that transgenic delivery of PON1 via bone marrow transplantation provided an effective anti-atherogenic gene therapy for mice lacking LDL receptors.

Throughout this application various publications have been referenced. The disclosures of these publications in their entireties are hereby incorporated by reference in this application in order to more fully describe the state of the art to which this invention pertains.

Although the invention has been described with reference to the examples provided above, it should be understood that various modifications can be made without departing from the spirit of the invention. Accordingly, the invention is limited only by the claims.

What is claimed is:

1. A nucleic acid comprising a nucleotide sequence encoding an inhibitor of a pro-atherogenic molecule operationally linked to a macrophage-specific expression element.

2. The nucleic acid of claim 1, wherein said inhibitor of a pro-atherogenic molecule is selected from the group consisting of a paraoxonase polypeptide, cholesterol-7α-hydroxylase polypeptide, apolipoprotein A1, or a functional fragment thereof.

3. The nucleic acid sequence of claim 1, wherein said macrophage-specific expression element comprises a macrophage-specific promoter or a macrophage-specific enhancer.

4. The nucleic acid sequence of claim 3, wherein said macrophage-specific expression element comprises a class A scavenger receptor promoter or enhancer.

5. A vector comprising the nucleic acid of claim 1.

6. An embryonic stem cell comprising the nucleic acid of claim 1.

7. An isolated mammalian cell comprising the nucleic acid of claim 1.

8. The mammalian cell of claim 7, wherein said cell is a mouse cell.

9. A recombinant cell comprising a macrophage expressing a nucleic acid encoding an inhibitor of a pro-atherogenic molecule.

10. The recombinant cell of claim 9, wherein said inhibitor of a pro-atherogenic molecule is selected from the group consisting of a paraoxonase polypeptide, cholesterol-7α-hydroxylase polypeptide, apolipoprotein A1, or a functional fragment thereof.

11. The recombinant cell of claim 9, wherein said macrophage is derived from a monocyte.

12. The recombinant cell of claim 9, wherein said macrophage is derived from a stem cell.

13. The recombinant cell of claim 9, further comprising a macrophage-specific expression element regulating expression of said nucleic acid encoding an inhibitor of a pro-atherogenic molecule.

14. The recombinant cell of claim 13, wherein said macrophage-specific expression element comprises a macrophage-specific promoter or a macrophage-specific enhancer.

15. The recombinant cell of claim 14, wherein said macrophage-specific expression element comprises a class A scavenger receptor promoter or enhancer.

16. The recombinant cell of claim 9, wherein said cell is derived from a mammalian cell.

17. The recombinant cell of claim 16, wherein said mammalian cell is derived from a human.

18. The recombinant cell of claim 16, wherein said mammalian cell is derived from a mouse.

19. The recombinant cell of claim 9, wherein said cell is isolated.

20. A transgenic non-human mammal comprising recombinant cells containing a transgenic nucleic acid encoding an inhibitor of a pro-atherogenic molecule.

21. The transgenic non-human mammal of claim 20, wherein said inhibitor of a pro-atherogenic molecule is selected from the group consisting of a paraoxonase polypeptide, cholesterol-7α-hydroxylase polypeptide, apolipoprotein A1, or a functional fragment thereof.

22. The transgenic non-human mammal of claim 20, wherein said non-human mammal is a mouse.

23. The transgenic mouse of claim 20, wherein said mouse is a C57BL/6J strain mouse.

24. The transgenic non-human mammal of claim 20, wherein said non-human mammal exhibits reduced susceptibility to developing atherosclerosis.

25. The transgenic non-human mammal of claim 20, wherein expression of said inhibitor of a pro-atherogenic molecule is regulated by a macrophage-specific expression element.

26. The transgenic non-human mammal of claim 25, wherein said macrophage-specific expression element comprises a macrophage-specific promoter or a macrophage-specific enhancer.

27. The transgenic non-human mammal of claim 26, wherein said macrophage-specific expression element comprises a class A scavenger receptor promoter or enhancer.

28. The transgenic non-human mammal of claim 22, wherein said mouse is homozygous for said nucleic acid expressing an inhibitor of a pro-atherogenic molecule.

29. The transgenic non-human mammal of claim 22, wherein said mouse is heterozygous for said nucleic acid expressing an inhibitor of a pro-atherogenic molecule.

30. A non-human mammalian cell isolated from the transgenic non-human mammal of claim 20.

31. The non-human mammalian cell of claim 30, wherein said cell is derived from a mouse.

32. The non-human mammalian cell of claim 20, wherein said cell is derived from a monocyte.

33. The non-human mammalian cell of claim 20, wherein said cell is derived from a macrophage.

34. A method for inhibiting or reducing atherosclerosis comprising administering to an individual a population of recombinant cells expressing a nucleic acid encoding an inhibitor of a pro-atherogenic molecule.

35. The method of claim 34, wherein said inhibitor of a pro-atherogenic molecule is selected from the group consisting of a paraoxonase polypeptide, cholesterol-7α-hydroxylase polypeptide, apolipoprotein A1, or a functional fragment thereof.

36. The method of claim 34, wherein said population of recombinant cells is derived from leukocytes.

37. The method of claim 34, wherein said population of recombinant cells is derived from monocytes.

38. The method of claim 34, wherein said population of recombinant cells is derived from macrophages.

39. The method of claim 34, wherein said population is derived from stem cells.

40. The method of claim 34, wherein expression of said inhibitor of a pro-atherogenic molecule is regulated by a macrophage-specific expression element.

41. The method of claim 40, wherein said macrophage-specific expression element comprises a macrophage-specific promoter or a macrophage-specific enhancer.

42. The transgenic non-human mammal of claim 41, wherein said macrophage-specific expression element comprises a class A scavenger receptor promoter or enhancer.

43. A method for inhibiting or reducing atherosclerosis comprising administering to an individual a nucleic acid encoding an inhibitor of a pro-atherogenic molecule, said inhibitor of a pro-atherogenic molecule operationally linked to a macrophage-specific expression element.

44. The method of claim 43, wherein said inhibitor of a pro-atherogenic molecule is selected from the group consisting of a paraoxonase polypeptide, cholesterol-7α-hydroxylase polypeptide, apolipoprotein A1, or a functional fragment thereof.

45. The method of claim 43, wherein said macrophage-specific expression element is a class A scavenger receptor promoter or enhancer.

46. The method of claim 43, wherein said cell is derived from a leukocyte.

47. The method of claim 43, wherein said cell is derived from a monocyte.

48. The method of claim 43, wherein said cell is derived from a macrophage.
